(19) United States
(12) Patent Application Publication
(10) Pub. No.: US 2026/0078163 A1
(43) Pub. Date: Mar. 19, 2026

(54) T-CELL RECEPTOR THAT TARGETS EGFR MUTATION AND METHODS OF USING THE SAME

(71) Applicant: BOARD OF REGENTS, THE UNIVERSITY OF TEXAS SYSTEM, Austin, TX (US)

(72) Inventors: Alexandre REUBEN, Missouri City, TX (US); Minying ZHANG, Houston, TX (US); John HEYMACH, Houston, TX (US); Greg LIZEE, Houston, TX (US); Peixin JIANG, Pearland, TX (US); Patrick HWU, Tampa, FL (US)

(21) Appl. No.: 19/109,491

(22) PCT Filed: Sep. 6, 2023
(86) PCT No.: PCT/US2023/073571

§ 371 Date: Mar. 6, 2025

Related U.S. Application Data
(60) Provisional application No. 63/374,704, filed on Sep. 6, 2022.

(51) Int. Cl. C07K 14/725; A61K 35/17; A61K 38/00; A61K 40/11; A61K 40/15; A61K 40/32; A61K 40/42; A61P 35/00; C12N 5/00; C12N 5/0783; C12N 15/11; C12N 15/63

(52) U.S. Cl. CPC C07K 14/7051; A61K 35/17; A61K 38/00; A61K 40/11; A61K 40/15; A61K 40/32; A61K 40/4204; A61P 35/00; C12N 5/0087; C12N 5/0636; C12N 5/0646; C12N 15/11; C12N 15/63; A61K 2239/55; C07K 2317/24; C07K 2317/31; C07K 2317/565

(57) ABSTRACT

The present disclosure relates to an engineered T-cell Receptors (TCRs) capable of binding to an epitope of an epidermal growth factor receptor (EGFR) comprising a T790M mutation presented on human leukocyte antigen-A (HLA-A)*02:01, wherein the epitope comprises the amino acid sequence of MQLMPFGCLL (SEQ ID NO: 1). Also disclosed are Tcell and BiTE therapies comprising the same, and methods of treating EGFR-TKI resistant lung cancers using the same.

### CROSS REFERENCE TO RELATED APPLICATIONS

The present application claims the priority benefit of U.S. Provisional Application No. 63/374,704, filed Sep. 6, 2022, which is hereby incorporated by reference in its entirety.

### REFERENCE TO SEQUENCE LISTING SUBMITTED ELECTRONICALLY VIA EFS-WEB

The content of the electronically submitted sequence listing in .XML file (Name: 4443_021PC01_Seglisting_ST26.xml; Size: 68,841 bytes; and Date of Creation: Aug. 15, 2023), filed with the application, is incorporated herein by reference in its entirety.

### FIELD OF THE DISCLOSURE

The disclosure generally relates to the fields of immunology, cellular therapy, and cancer therapy. For example, the present disclosure provides engineered T-cell Receptors (TCRs), T-cell therapies comprising the same, and methods of using the same.

### BACKGROUND

Lung cancer is the second most commonly diagnosed cancer in the United States. Non-small cell lung cancers (NSCLCs) are the most common type of lung cancer, estimated to account for over 195,000 diagnoses (see, e.g., www.cancer.net/cancer-types/lung-cancer-non-small-cell/statistics). Standard therapy for NSCLCs uses epidermal growth factor receptor (EGFR) tyrosine kinase inhibitors (EGFR-TKIs). Treatment with EGFR-TKIs, such as gefitinib and erlotinib, has achieved high clinical response rates in patients with NSCLCs. However, over time, most treated NSCLC tumors develop acquired resistance to EGFR-TKIs, which is associated with an EGFR T790M resistance mutation in about half the cases. Patients having a T790M point mutation of EGFR are resistant to first and second generation EGFR-TKI treatments. About 20% of advanced-stage NSCLC patients have the T790M point mutation. Currently, there are no effective treatment options for patients with this resistance mutation. As such, there is a need for development of effective therapies for the population of NSCLC patients with the EGFR-TKI resistance, particularly those having the T790M mutation.

### BRIEF SUMMARY

The present disclosure provides T-cell receptors (TCRs) capable of binding to an epitope of an epidermal growth factor receptor (EGFR) comprising a T790M mutation presented on human leukocyte antigen-A (HLA-A)*02:01 and T-cell therapies comprising the same. In some aspects, the epitope comprises the amino acid sequence of MQLMPFGCLL (SEQ ID NO: 1).

Certain aspects of the disclosure are directed to an engineered T-cell Receptor (TCR) comprising: (a) a TCR beta chain comprising (i) a variable TCR beta chain complementarity determining region 1 (CDR1β) comprising the amino acid sequence set forth in SEQ ID NO: 5, (ii) a CDR2β comprising the amino acid sequence set forth in SEQ ID NO: 6, and (iii) a CDR3β comprising the amino acid sequence set forth in SEQ ID NO: 7 or SEQ ID NO:43; and (b) a TCR alpha chain comprising (i) a variable TCR alpha chain complementarity determining region 1 (CDR1α) comprising the amino acid sequence set forth in SEQ ID NO: 2, (ii) a CDR2α comprising the amino acid sequence set forth in SEQ ID NO: 3, and (iii) a CDR3α comprising the amino acid sequence set forth in SEQ ID NO: 4. In some aspects, the engineered TCR is capable of binding to an epitope of an epidermal growth factor receptor (EGFR) comprising a T790M mutation presented on a human leukocyte antigen-A (HLA-A)*02:01 antigen, wherein the epitope comprises the amino acid sequence of MQLMPFGCLL (SEQ ID NO: 1).

In some aspects, the TCR beta chain comprises any one of SEQ ID NO: 16, 32, or 46.

In some aspects, the TCR alpha chain comprises any one of SEQ ID NO: 14, 30, or 44.

Certain aspects of the disclosure are directed to an engineered T-cell Receptor (TCR) comprising: (a) a TCR beta chain comprising (i) a variable TCR beta chain complementarity determining region 1 (CDR1β) of SEQ ID NO: 16, 32, or 46, (ii) a CDR2β of SEQ ID NO: 16, 32, or 46, and (iii) a CDR3B of SEQ ID NO: 16, 32, or 46; and a TCR alpha chain comprising (i) a variable TCR alpha chain complementarity determining region 1 (CDR1α) of SEQ ID NO: 14, 30, or 44, (ii) a CDR2α of SEQ ID NO: 14, 30, or 44, and (iii) a CDR3α of SEQ ID NO: 14, 30, or 44, wherein the engineered TCR binds an epitope of an epidermal growth factor receptor (EGFR) comprising a T790M mutation presented on a human leukocyte antigen-A (HLA-A)*02:01, wherein the epitope comprises the amino acid sequence of MQLMPFGCLL (SEQ ID NO: 1).

Certain aspects of the disclosure are directed to an engineered TCR comprising: (a) a TCR beta chain comprising (i) a variable TCR beta chain complementarity determining region 1 (CDR1β) comprising the amino acid sequence set forth in SEQ ID NO: 5, (ii) a CDR2β chain comprising the amino acid sequence set forth in SEQ ID NO: 6, and (iii) a CDR3β comprising the amino acid sequence set forth in SEQ ID NO: 7; and a TCR alpha chain comprising (i) a variable TCR alpha chain complementarity determining region 1 (CDR1α) comprising the amino acid sequence set forth in SEQ ID NO: 2, (ii) a CDR2α comprising the amino acid sequence set forth in SEQ ID NO: 3, and (iii) a CDR3α comprising the amino acid sequence set forth in SEQ ID NO: 4.

In some aspects, the TCR beta chain comprises (i) the CDR1β comprising the amino acid sequence set forth in SEQ ID NO: 5, (ii) the CDR2β comprising the amino acid sequence set forth in SEQ ID NO: 6, and (iii) the CDR3β comprising the amino acid sequence set forth in SEQ ID NO: 34; and the TCR alpha chain comprises (i) the CDR1α comprising the amino acid sequence set forth in SEQ ID NO: 2, (ii) the CDR2α comprising the amino acid sequence set forth in SEQ ID NO: 3, and (iii) the CDR3α comprising the amino acid sequence set forth in SEQ ID NO: 35.

In some aspects, the TCR beta chain comprises (i) the CDR1β comprising the amino acid sequence set forth in SEQ ID NO: 5, (ii) the CDR2β comprising the amino acid sequence set forth in SEQ ID NO: 6, and (iii) the CDR3β comprising the amino acid sequence set forth in SEQ ID NO: 43; and the TCR alpha chain comprises (i) the CDR1α comprising the amino acid sequence set forth in SEQ ID NO: 2, (ii) the CDR2α comprising the amino acid sequence set forth in SEQ ID NO: 3, and (iii) the CDR3α comprising the amino acid sequence set forth in SEQ ID NO: 4.

In some aspects, the TCR beta chain comprises (i) the CDR1β comprising the amino acid sequence set forth in SEQ ID NO: 5, (ii) the CDR2β comprising the amino acid sequence set forth in SEQ ID NO: 6, and (iii) the CDR3β comprising the amino acid sequence set forth in SEQ ID NO: 36; and the TCR alpha chain comprises (i) the CDR1α comprising the amino acid sequence set forth in SEQ ID NO: 2, (ii) the CDR2α comprising the amino acid sequence set forth in SEQ ID NO: 3, and (iii) the CDR3α comprising the amino acid sequence set forth in SEQ ID NO: 37.

In some aspects, the TCR beta chain comprises (i) the CDR1β comprising the amino acid sequence set forth in SEQ ID NO: 5, (ii) the CDR2β comprising the amino acid sequence set forth in SEQ ID NO: 6, and (iii) the CDR3β comprising the amino acid sequence set forth in SEQ ID NO: 38; and the TCR alpha chain comprises (i) the CDR1α comprising the amino acid sequence set forth in SEQ ID NO: 2, (ii) the CDR2α comprising the amino acid sequence set forth in SEQ ID NO: 3, and (iii) the CDR3α comprising the amino acid sequence set forth in SEQ ID NO: 39.

In some aspects, the TCR beta chain comprises (i) the CDR1β comprising the amino acid sequence set forth in SEQ ID NO: 5, (ii) the CDR2β comprising the amino acid sequence set forth in SEQ ID NO: 6, and (iii) the CDR3β comprising the amino acid sequence set forth in SEQ ID NO: 40; and the TCR alpha chain comprises (i) the CDR1α comprising the amino acid sequence set forth in SEQ ID NO: 2, (ii) the CDR2α comprising the amino acid sequence set forth in SEQ ID NO: 3, and (iii) the CDR3α comprising the amino acid sequence set forth in SEQ ID NO: 41.

In some aspects, the TCR beta chain comprises (i) the CDR1β comprising the amino acid sequence set forth in SEQ ID NO: 5, (ii) the CDR2β comprising the amino acid sequence set forth in SEQ ID NO: 6, and (iii) the CDR3β comprising the amino acid sequence set forth in SEQ ID NO: 40; and the TCR alpha chain comprises (i) the CDR1α comprising the amino acid sequence set forth in SEQ ID NO: 2, (ii) the CDR2α comprising the amino acid sequence set forth in SEQ ID NO: 3, and (iii) the CDR3α comprising the amino acid sequence set forth in SEQ ID NO: 42.

In some aspects, the engineered TCR of the disclosure comprises an extracellular antigen-binding domain.

In some aspects, the extracellular region comprises an antigen-binding domain variable region and a constant region. In some aspects, the extracellular region further comprises a signal sequence.

In some aspects, the engineered TCR comprises an antigen-binding domain variable region comprising: i) a CDR1β, a CDR2β, and a CDR3β of SEQ ID NO: 16, 32, or 46; and ii) a CDR1α, CDR2α, and a CDR3α of SEQ ID NO: 14, 30, or 44.

In some aspects, the CDRs are the Kabat-defined CDRs, the Chothia-defined CDRs, the AbM-defined CDRs, or the IMGT-defined CDRs.

In some aspects, the engineered TCR comprises a beta chain (TCR-β) and an alpha chain (TCR-α), wherein the TCR-β comprises an amino acid sequence having at least 85%, at least 90%, at least 91% at least 92%, at least 93%, at least 94%, at least 95%, at least 96%, at least 97%, at least 98%, at least 99%, or at least 100% sequence identity to SEQ ID NO: 46 and the TCR-α comprises an amino acid sequence having at least 85%, at least 90%, at least 91%, at least 92%, at least 93%, at least 94%, at least 95%, at least 96%, at least 97%, at least 98%, at least 99%, or at least 100% sequence identity to SEQ ID NO: 44. In some aspects, the engineered TCR comprising a beta chain (TCR-β) and an alpha chain (TCR-α), wherein the TCR-β comprises an amino acid sequence having at least 85%, at least 90%, at least 91% at least 92%, at least 93%, at least 94%, at least 95%, at least 96%, at least 97%, at least 98%, at least 99%, or at least 100% sequence identity to SEQ ID NO: 32 and the TCR-α comprises an amino acid sequence having at least 85%, at least 90%, at least 91%, at least 92%, at least 93%, at least 94%, at least 95%, at least 96%, at least 97%, at least 98%, at least 99%, or at least 100% sequence identity to SEQ ID NO: 30.

In some aspects, the engineered TCR comprises a TCR-β chain comprising an amino acid sequence at least 85% identical to the amino acid sequence of SEQ ID NO: 16, SEQ ID NO:46, or SEQ ID NO: 32.

In some aspects, the engineered TCR comprises a TCR-β chain comprising an amino acid sequence at least 90% identical to the amino acid sequence of SEQ ID NO: 16, SEQ ID NO:46, or SEQ ID NO: 32.

In some aspects, the engineered TCR comprises a TCR-β chain comprising an amino acid sequence at least 95% identical to the amino acid sequence of SEQ ID NO: 16, SEQ ID NO:46, or SEQ ID NO: 32.

In some aspects, the engineered TCR comprises a TCR-β chain comprising an amino acid sequence at least 98% identical to the amino acid sequence of SEQ ID NO: 16, SEQ ID NO:46, or SEQ ID NO: 32.

In some aspects, the engineered TCR comprises a TCR-β chain comprising an amino acid sequence at least 99% identical to the amino acid sequence of SEQ ID NO: 16, SEQ ID NO:46, or SEQ ID NO: 32.

In some aspects, the engineered TCR comprises a TCR-β chain comprising an amino acid sequence identical to the amino acid sequence of SEQ ID NO: 16, SEQ ID NO:46, or SEQ ID NO: 32.

In some aspects, the engineered TCR comprises a TCR-α chain comprising an amino acid sequence at least 85% identical to the amino acid sequence of SEQ ID NO: 14, SEQ ID NO:44, or SEQ ID NO: 30.

In some aspects, the engineered TCR comprises a TCR-α chain comprising an amino acid sequence at least 90% identical to the amino acid sequence of SEQ ID NO: 14, SEQ ID NO:44, or SEQ ID NO: 30.

In some aspects, the engineered TCR comprises a TCR-α chain comprising an amino acid sequence at least 95% identical to the amino acid sequence of SEQ ID NO: 14, SEQ ID NO:44, or SEQ ID NO: 30.

In some aspects, the engineered TCR comprises a TCR-α chain comprising an amino acid sequence at least 98% identical to the amino acid sequence of SEQ ID NO: 14, SEQ ID NO:44, or SEQ ID NO: 30.

In some aspects, the engineered TCR comprises a TCR-α chain comprising an amino acid sequence at least 99% identical to the amino acid sequence of SEQ ID NO: 14, SEQ ID NO:44, or SEQ ID NO: 30.

In some aspects, the engineered TCR comprises a TCR-α chain comprising an amino acid sequence identical to the amino acid sequence of SEQ ID NO: 14, SEQ ID NO:44, or SEQ ID NO: 30.

In some aspects, the engineered TCR comprises an amino acid sequence having at least 85%, at least 90%, at least 91%, at least 92%, at least 93%, at least 94%, at least 95%, at least 96%, at least 97%, at least 98%, at least 99%, or at least 100% sequence identity to SEQ ID NO: 28.

In some aspects, the engineered TCR is humanized or chimeric.

Certain aspects of the disclosure are directed to a polynucleotide comprising a nucleic acid encoding a TCR-β chain or antigen-binding portion thereof disclosed herein (e.g., SEQ ID NO: 16, SEQ ID NO: 46, or SEQ ID NO: 32).

Certain aspects of the disclosure are directed to polynucleotide comprising a nucleic acid encoding a TCR-α chain or antigen-binding portion thereof disclosed herein (e.g., SEQ ID NO: 14, SEQ ID NO: 44, or SEQ ID NO: 30).

Certain aspects of the disclosure are directed to a polynucleotide comprising a nucleic acid encoding a TCR-β chain or antigen-binding portion thereof and a TCR-α chain or antigen-binding portion thereof disclosed herein. In some aspects, the TCR-β chain and/or the TCR-α chain comprises a signal sequence. In some aspects, the polynucleotide further comprises a linker located between the nucleic acid encoding a TCR-β chain and the nucleic acid encoding a TCR-α chain disclosed herein.

In some aspects, the TCR-β comprises an antigen-binding domain variable region which is encoded by a polynucleotide sequence comprising a nucleic acid sequence having at least 85%, at least 90%, at least 91%, at least 92%, at least 93%, at least 94%, at least 95%, at least 96%, at least 97%, at least 98%, at least 99%, or at least 100% sequence identity to SEQ ID NO: 17.

In some aspects, the TCR-α comprises an antigen-binding domain variable region which is encoded by a polynucleotide sequence comprising a nucleic acid sequence having at least 85%, at least 90%, at least 91%, at least 92%, at least 93%, at least 94%, at least 95%, at least 96%, at least 97%, at least 98%, at least 99%, or at least 100% sequence identity to SEQ ID NO: 15.

In some aspects, the TCR-β comprises an antigen-binding domain variable region and a constant region which are encoded by a polynucleotide sequence comprising a nucleic acid sequence having at least 85%, at least 90%, at least 91%, at least 92%, at least 93%, at least 94%, at least 95%, at least 96%, at least 97%, at least 98%, at least 99%, or at least 100% sequence identity to SEQ ID NO: 47.

In some aspects, the TCR-α comprises a antigen-binding domain variable region and a constant region which are encoded by a polynucleotide sequence comprising a nucleic acid sequence having at least 85%, at least 90%, at least 91%, at least 92%, at least 93%, at least 94%, at least 95%, at least 96%, at least 97%, at least 98%, at least 99%, or at least 100% sequence identity to SEQ ID NO: 45.

In some aspects, the TCR-β comprises a signal sequence, antigen-binding domain variable region and constant region which are encoded by a polynucleotide sequence comprising a nucleic acid sequence having at least 85%, at least 90%, at least 91%, at least 92%, at least 93%, at least 94%, at least 95%, at least 96%, at least 97%, at least 98%, at least 99%, or at least 100% sequence identity to SEQ ID NO: 33.

In some aspects, the TCR-α comprises a signal sequence, antigen-binding domain variable region and constant region which are encoded by a polynucleotide sequence comprising a nucleic acid sequence having at least 85%, at least 90%, at least 91%, at least 92%, at least 93%, at least 94%, at least 95%, at least 96%, at least 97%, at least 98%, at least 99%, or at least 100% sequence identity to SEQ ID NO: 31.

In some aspects, the nucleic acid sequence encoding the engineered TCR comprises a sequence having at least 85%, at least 90%, at least 91% at least 92%, at least 93%, at least 94%, at least 95%, at least 96%, at least 97%, at least 98%, at least 99%, or at least 100% sequence identity to SEQ ID NO: 29.

Certain aspects of the disclosure are directed to a vector comprising a polynucleotide disclosed herein.

Certain aspects of the disclosure are directed to a host cell comprising a polynucleotide disclosed herein.

In some aspects, the host cell is selected from the group consisting of CHO, HEK-293T, HeLa and BHK cells, optionally wherein the CHO cell is a CHO-K1SP cell.

Certain aspects of the disclosure are directed to a T-cell comprising an engineered TCR disclosed herein (TCR-T cell). In some aspects, the T cell is a CD4+ T cell, CD8+ cell, or CD4+/CD8+ T cell. In some aspects, the T cell is a natural killer (NK) cell.

In some aspects, the T cells are isolated from peripheral blood mononuclear cells (PBMCs).

Certain aspects of the disclosure are directed to a bispecific T cell engager (BiTE), wherein the BiTE comprises an engineered TCR disclosed herein.

Certain aspects of the disclosure are directed to a method of treating cancer or a tumor in a subject, the method comprising administering to the subject a therapeutically effective amount of the engineered TCR, a TCR-T cell, or the BiTE disclosed herein.

In some aspects, the subject is a human.

In some aspects, the cancer is lung cancer.

In some aspects, the lung cancer is non-small cell lung cancer (NSCLC).

In some aspects, the cancer or tumor comprises an epidermal growth factor receptor (EGFR) T790M mutation on human leukocyte antigen-A (HLA-A)*02:01.

In some aspects, the cancer or tumor is epidermal growth factor receptor (EGFR) tyrosine kinase inhibitor (EGFR-TKI) resistant.

### DESCRIPTION OF FIGURES

FIG. 1A shows flow cytometry analysis indicating the proportion of epidermal growth factor receptor (EGFR) comprising a T790M mutation on human leukocyte antigen-A (HLA-A)*02:01 antigen-specific T cells from healthy donor blood following first stimulation.

FIG. 1B shows flow cytometry analysis indicating the proportion of epidermal growth factor receptor (EGFR) comprising a T790M mutation on human leukocyte antigen-A (HLA-A)*02:01 antigen-specific T cells from healthy donor blood following a second stimulation.

FIG. 2 shows a bar plot of unpulsed, endogenous, and pulsed T cells % killing of target tumor cells.

FIG. 3 shows isolated sequences of T-cell receptor (TCR) alpha and beta chain CDR3s.

FIG. 4A shows TCR transduction into donor peripheral blood mononuclear cells (PBMCs).

FIG. 4B shows the expression of transduced TCR following cell sorting.

FIG. 5 shows the ability of transduced T cell population to kill targeted tumor cells expressing the antigen of interest.

### DETAILED DESCRIPTION

Certain aspects of the disclosure are directed to an isolated, recombinant, or engineered T cell receptors (TCRs), which comprise an antigen recognition portion that recognizes a specific tumor associated antigen (TAA) of interest. In some aspects, the TAA comprises an epitope of an epidermal growth factor receptor (EGFR) comprising a T790M mutation presented on a human leukocyte antigen-A (HLA-A)*02:01. In some aspects, the epitope comprises the amino acid sequence of MQLMPFGCLL (SEQ ID NO: 1). In some aspects, the TCR is derived from a T cell that specifically binds an antigen comprising a T790M mutation presented on a human leukocyte antigen-A (HLA-A)*02:01 (e.g., SEQ ID NO: 1).

In some aspects, the TCR (e.g., engineered TCR comprising a TCR-β chain and TCR-α disclosed herein) is capable of binding to an epitope of an EGFR comprising a T790M mutation presented on a human leukocyte antigen-A (HLA-A)*02:01, wherein the TCR comprises: (a) a TCR beta chain comprising (i) a variable TCR beta chain complementarity determining region 1 (CDR1β) comprising the amino acid sequence set forth in SEQ ID NO:5, (ii) a CDR2β chain comprising the amino acid sequence set forth in SEQ ID NO: 6, and (iii) a CDR3β comprising the amino acid sequence set forth in SEQ ID NO: 7; and (b) a TCR alpha chain comprising (i) a variable TCR alpha chain complementarity determining region 1 (CDR1α) comprising the amino acid sequence set forth in SEQ ID NO: 3, (ii) a CDR2α comprising the amino acid sequence set forth in SEQ ID NO:4, and (iii) a CDR3α comprising the amino acid sequence set forth in SEQ ID NO: 5.

In some aspects, the TCR beta chain comprises (i) the CDR1β comprising the amino acid sequence set forth in SEQ ID NO: 5, (ii) the CDR2β comprising the amino acid sequence set forth in SEQ ID NO: 6, and (iii) the CDR3β comprising the amino acid sequence set forth in SEQ ID NO: 34; and the TCR alpha chain comprises (i) the CDR1α comprising the amino acid sequence set forth in SEQ ID NO: 2, (ii) the CDR2α comprising the amino acid sequence set forth in SEQ ID NO: 3, and (iii) the CDR3α comprising the amino acid sequence set forth in SEQ ID NO: 35.

In some aspects, the TCR beta chain comprises (i) the CDR1β comprising the amino acid sequence set forth in SEQ ID NO: 5, (ii) the CDR2β comprising the amino acid sequence set forth in SEQ ID NO: 6, and (iii) the CDR3β comprising the amino acid sequence set forth in SEQ ID NO: 43; and the TCR alpha chain comprises (i) the CDR1α comprising the amino acid sequence set forth in SEQ ID NO: 2, (ii) the CDR2α comprising the amino acid sequence set forth in SEQ ID NO: 3, and (iii) the CDR3α comprising the amino acid sequence set forth in SEQ ID NO: 4.

In some aspects, the TCR beta chain comprises (i) the CDR1β comprising the amino acid sequence set forth in SEQ ID NO: 5, (ii) the CDR2β comprising the amino acid sequence set forth in SEQ ID NO: 6, and (iii) the CDR3β comprising the amino acid sequence set forth in SEQ ID NO: 36; and the TCR alpha chain comprises (i) the CDR1α comprising the amino acid sequence set forth in SEQ ID NO: 2, (ii) the CDR2α comprising the amino acid sequence set forth in SEQ ID NO: 3, and (iii) the CDR3α comprising the amino acid sequence set forth in SEQ ID NO: 37.

In some aspects, the TCR beta chain comprises (i) the CDR1β comprising the amino acid sequence set forth in SEQ ID NO: 5, (ii) the CDR2β comprising the amino acid sequence set forth in SEQ ID NO: 6, and (iii) the CDR3β comprising the amino acid sequence set forth in SEQ ID NO: 38; and the TCR alpha chain comprises (i) the CDR1α comprising the amino acid sequence set forth in SEQ ID NO: 2, (ii) the CDR2α comprising the amino acid sequence set forth in SEQ ID NO: 3, and (iii) the CDR3α comprising the amino acid sequence set forth in SEQ ID NO: 39.

In some aspects, the TCR beta chain comprises (i) the CDR1β comprising the amino acid sequence set forth in SEQ ID NO: 5, (ii) the CDR2β comprising the amino acid sequence set forth in SEQ ID NO: 6, and (iii) the CDR3β comprising the amino acid sequence set forth in SEQ ID NO: 40; and the TCR alpha chain comprises (i) the CDR1α comprising the amino acid sequence set forth in SEQ ID NO: 2, (ii) the CDR2α comprising the amino acid sequence set forth in SEQ ID NO: 3, and (iii) the CDR3α comprising the amino acid sequence set forth in SEQ ID NO: 41.

In some aspects, the TCR beta chain comprises (i) the CDR1β comprising the amino acid sequence set forth in SEQ ID NO: 5, (ii) the CDR2β comprising the amino acid sequence set forth in SEQ ID NO: 6, and (iii) the CDR3β comprising the amino acid sequence set forth in SEQ ID NO: 40; and the TCR alpha chain comprises (i) the CDR1α comprising the amino acid sequence set forth in SEQ ID NO: 2, (ii) the CDR2α comprising the amino acid sequence set forth in SEQ ID NO: 3, and (iii) the CDR3α comprising the amino acid sequence set forth in SEQ ID NO: 42.

In some aspects, the engineered TCR comprises: i) a CDR1β, a CDR2β, and a CDR3β of SEQ ID NO: 16; and ii) a CDR1α, CDR2α, and a CDR3α of SEQ ID NO: 14. In some aspects, the CDRs are the Kabat-defined CDRs, the Chothia-defined CDRs, the AbM-defined CDRs, or the IMGT-defined CDRs.

In some aspects, the engineered TCR comprises a beta chain (TCR-β) and an alpha chain (TCR-α), wherein the TCR-β comprises an amino acid sequence having at least 85%, at least 90%, at least 91% at least 92%, at least 93%, at least 94%, at least 95%, at least 96%, at least 97%, at least 98%, at least 99%, or at least 100% sequence identity to SEQ ID NO: 16 and the TCR-α comprises an amino acid sequence having at least 85%, at least 90%, at least 91%, at least 92%, at least 93%, at least 94%, at least 95%, at least 96%, at least 97%, at least 98%, at least 99%, or at least 100% sequence identity to SEQ ID NO: 14.

In some aspects, the engineered TCR comprises a beta chain (TCR-β) and an alpha chain (TCR-α), wherein the TCR-β comprises an amino acid sequence having at least 85%, at least 90%, at least 91% at least 92%, at least 93%, at least 94%, at least 95%, at least 96%, at least 97%, at least 98%, at least 99%, or at least 100% sequence identity to SEQ ID NO: 46 and the TCR-α comprises an amino acid sequence having at least 85%, at least 90%, at least 91%, at least 92%, at least 93%, at least 94%, at least 95%, at least 96%, at least 97%, at least 98%, at least 99%, or at least 100% sequence identity to SEQ ID NO: 44.

In some aspects, the engineered TCR-β comprises an amino acid sequence having at least 85%, at least 90%, at least 91% at least 92%, at least 93%, at least 94%, at least 95%, at least 96%, at least 97%, at least 98%, at least 99%, or at least 100% sequence identity to SEQ ID NO: 32; and the TCR-α comprises an amino acid sequence having at least 85%, at least 90%, at least 91%, at least 92%, at least 93%, at least 94%, at least 95%, at least 96%, at least 97%, at least 98%, at least 99%, or at least 100% sequence identity to SEQ ID NO: 30.

Certain aspects of the disclosure are directed to a T-cell (TCR-T cell) or a bispecific T cell engager (BiTE) comprising an engineered TCR disclosed herein.

Certain aspects of the disclosure are directed to a method of treating cancer or a tumor (e.g., non-small cell lung cancer (NSCLC)) in a subject, the method comprising administering to the subject a therapeutically effective amount of the engineered TCR, a TCR-T cell, or the BiTE disclosed herein.

In some aspects, the cancer or tumor comprises an epidermal growth factor receptor (EGFR) T790M mutation on human leukocyte antigen-A (HLA-A)*02:01. In some aspects, the cancer or tumor is epidermal growth factor receptor (EGFR) tyrosine kinase inhibitor (EGFR-TKI) resistant.

### I. Definitions

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those disclosed herein can be used in practice or testing of the present disclosure, suitable methods and materials are disclosed below. The materials, methods and examples are illustrative only and are not intended to be limiting. Other features and advantages of the disclosure will be apparent from the detailed description and from the claims.

The following terms and definitions are provided.

The singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

The term “at least” prior to a number or series of numbers is understood to include the number adjacent to the term “at least,” and all subsequent numbers or integers that could logically be included, as clear from context. For example, the number of nucleotides in a nucleic acid molecule must be an integer. For example, “at least 18 nucleotides of a 21-nucleotide nucleic acid molecule” means that 18, 19, 20, or 21 nucleotides have the indicated property. When at least is present before a series of numbers or a range, it is understood that “at least” can modify each of the numbers in the series or range. “At least” is also not limited to integers (e.g., “at least 5%” includes 5.0%, 5.1%, 5.18% without consideration of the number of significant figures).

Throughout this disclosure, various aspects of this disclosure are presented in a range format. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “T cell receptor” or “TCR”, also referred to herein as a “TCR protein”, “TCR portion” or “TCR unit” is a protein complex found on the surface of all T cells. In some aspects, a TCR can bind to CD3 by a non-covalent bond to form a TCR-CD3 complex. TCRs can recognize antigens bound to major histocompatibility complex molecules. TCRs are heterodimers including two different peptide chains. There are two categories of TCRs: TCR1 and TCR2. TCR1 is composed of peptide chains gamma (γ) and delta (6); and TCR2 is composed of peptide chains alpha (a) and beta (0). Each peptide chain can be divided into several parts, such as variable region (V region), constant region (C region), transmembrane region and cytoplasmic region, characterized in that the cytoplasmic region is typically short in length. TCR molecules are immunoglobulins, and their antigen specificity resides in the V regions (e.g., Va and Vp), each of which has three hypervariable regions CDR1, CDR2, and CDR3, in which CDR3 has the largest variation and determines the binding specificity of the TCR to an antigen. When a TCR recognizes a MHC-antigen peptide complex, CDR1 and CDR2 recognize and bind to the side wall of the antigen binding groove of the MHC molecule, and CDR3 binds to the antigen peptide. In some aspects, a partial genetic modification can be used to improve the “affinity” and effectiveness of TCRs to target, thereby providing a high-affinity TCR. Unless otherwise stated, the term “TCR” should be understood to encompass functional TCR fragments thereof. The term also encompasses intact or full-length TCRs, including TCRs in the ap form or 76 form.

As used herein, an “engineered TCR” or “recombinant TCR” refers to a recombinant protein derived from or comprising one or more of the same or similar structural properties (e.g., antigen binding specificity) as a TCR protein of an antigen-specific T cell. In some aspects, the TCR of the antigen-specific T cell specifically recognizes a tumor antigen of interest (e.g., an epidermal growth factor receptor (EGFR) comprising a T790M mutation on human leukocyte antigen-A (HLA-A)*02:01).

As used herein, a “TCR T cell” or “engineered TCR T cell” is a T cell that has been transduced with (for example, according to the methods disclosed herein) and expresses an engineered TCR disclosed herein. In some aspects, the T cell is a CD4+ T cell, CD8+ T cell, or CD4+/CD8+ T cell. In some aspects, the T cell is a natural killer (NK) cell.

The term “effector function” refers to the specialized function of a cell. For example, the effector function of T cells can be cytolytic activity or auxiliary activity, including the secretion of cytokines.

The term “intracellular signaling domain” refers to a part of a protein that transduces effector function signals and guides cells to perform specialized functions. Although the entire intracellular signaling domain can usually be used, in many cases it is not necessary to use the entire chain. If the truncated part of the intracellular signaling domain is used, such a truncated part can be used instead of the complete chain as long as it transduces the effector function signal. Therefore, the term “intracellular signaling domain” is intended to include any truncated portion of the intracellular signaling domain sufficient to transduce effector function signals.

Examples of intracellular signaling domains include cytoplasmic sequences of T cell receptors (TCRs) and co-receptors that act synergistically to initiate signal transduction after antigen receptor engagement, and derivatives or variants of any of these sequences and any recombinant sequence with the same functional capabilities. T cell activation can be mediated by two different types of cytoplasmic signaling sequences: the signaling sequence (primary intracellular signal transduction domain) that initiates antigen-dependent primary activation through TCR and the signaling sequence acts in an antigen-independent manner to provide secondary or costimulatory signals (secondary cytoplasmic domains, such as costimulatory domains). The primary signaling domain modulates the primary activation of the TCR complex in a stimulating manner or in an inhibitory manner. Primary intracellular signaling domains that act in a stimulating manner can contain signaling motifs, which are called immunoreceptor tyrosine-based activation motifs (ITAM).

Transmembrane domains can be derived from natural sources or recombinant sources. In the case of natural sources, the domain can be derived from any membrane-bound or transmembrane protein. The transmembrane domain can signal to the intracellular domain when an engineered TCR binds to a target. Useful transmembrane domains can include at least the following transmembrane regions: for example, the alpha, beta, or zeta chains of a T cell receptor, or CD28, CD3epsilon, CD45, CD4, CD5, CD8, CD9, CD16, CD22, CD33, CD37, CD64, CD80, CD86, CD134, CD137, CD154. In some cases, the transmembrane domain can be connected to the extracellular region of the engineered TCR (such as the antigen binding domain of the TCR).

As used herein, the term “immune effector cell” refers to a cell that exerts an effector function during an immune response, including, for example, immune cells secreting cytokines and/or chemokines, killing microorganisms, secreting antibodies, and recognizing or eliminating tumor cells. In some embodiments, immune effector cells include T cells (cytotoxic T cells, helper T cells, tumor-infiltrating T cells), B cells, natural killer cells, neutrophils, macrophages, and dendritic cells.

As used herein, the term “immune effector function” includes any function mediated by the composition of the immune system, which can lead to inhibition of tumor growth and/or inhibition of tumorigenesis, including inhibition the spread and metastasis of a tumor. Preferably, the immune effector function kills tumor cells.

As used herein, the term “antigen presenting cell” or “APC” refers to a cell in the immune system that display a complex of foreign antigens and major histocompatibility complex (MHC) on the surface, such as helper cells (such as B cells, dendritic cells, etc.). T cells can recognize these complexes using a T cell receptor (TCR) thereof. APC processes the antigen and presents it to T cells.

As used herein, the term “anti-tumor effect” refers to a biological effect that can be manifested in various ways, including but not limited to, for example, reduction in tumor volume, reduction in the number of tumor cells, reduction in the number of metastases, increase in life expectancy, reduction in tumor cell proliferation, and reduction in tumor cell survival rate, or improvement in various physiological symptoms related to cancerous conditions. The “anti-tumor effect” can also be expressed by the ability of the peptides, polynucleotides, cells and antibodies of the present disclosure to prevent or reduce the frequency of tumorigenesis.

As used herein, the term “autologous” refers to any material derived from an individual that will later be reintroduced into that same individual.

As used herein, the term “allogeneic” refers to any material derived from a different animal or a different patient of the same species as the individual into which the material is introduced. When the genes at one or more loci are different, two or more individuals are considered to be allogeneic to each other. In some aspects, allogeneic materials from individuals of the same species may be genetically different enough for antigenic interaction to occur.

As used herein, the term “genetically engineered cell” refers to a cell modified by means of genetic engineering.

As used herein, the term “stimulation” refers to a primary response induced by the binding of a stimulation domain or a stimulating molecule (e.g., TCR/CD3 complex) to its cognate ligand, thereby mediating signaling events, such as, but not limited to, signaling via TCR/CD3 complex. The stimulation can mediate changes in the expression of certain molecules and/or reorganization of the cytoskeleton structure.

As used herein, the term “stimulatory molecule” or “stimulatory domain” refers to a molecule or a part thereof expressed by T cells, which provides a primary cytoplasmic signaling sequence that modulates the primary activation of the TCR complex in a stimulating manner for at least some aspects of the T cell signaling pathway. In some aspects, the primary signal is initiated by, for example, the binding of the TCR/CD3 complex to the peptide-loaded MHC molecule, and it leads to the mediation of T cell responses including but not limited to proliferation, activation, differentiation, etc.

As used herein, the intracellular signaling domain may comprise a costimulatory intracellular domain. Exemplary costimulatory intracellular signaling domains include intracellular signaling domains derived from molecules responsible for costimulatory signals or antigen-independent stimulation.

As used herein, the term “costimulatory molecule” refers to a homologous binding partner on T cells, which specifically binds to a costimulatory ligand, thereby mediating the costimulatory response of T cells, such as but not limited to proliferation. Co-stimulatory molecules are cell surface molecules other than antigen receptors or their ligands required for an effective immune response. Costimulatory molecules include but are not limited to MHC class 1 molecules, BTLA and Toll ligand receptors, as well as DAP10, DAP12, CD30, LIGHT, OX40, CD2, CD27, CD28, CDS, ICAM-1, LFA-1 (CD11a/CD18) and 4-1BB (CD137). The costimulatory intracellular signaling domain can be the intracellular part of a costimulatory molecule. Costimulatory molecules can be represented in the following protein families: TNF receptor proteins, immunoglobulin-like proteins, cytokine receptors, integrins, signaling lymphocyte activation molecules (SLAM proteins), and activating NK cell receptors. Examples of such molecules include CD27, CD28, 4-1BB (CD137), OX40, GITR, CD30, CD40, ICOS, BAFFR, HVEM, lymphocyte function associated antigen 1 (LFA-1), CD2, CD7, LIGHT, NKG2C, SLAMF7, NKp80, CD160, B7-H3 and ligands that specifically bind to CD83.

As used herein, the costimulatory ligand provides a signal which, in addition to the primary signal provided by, for instance, binding of a TCR/CD3 complex with an MHC molecule loaded with peptide, mediates a T cell response. The T cell response includes, but is not limited to, proliferation activation, differentiation and the like. A costimulatory ligand can include but is not limited to CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, M1CB, HVEM, lymphotoxin beta receptor, 3/TR6, ILT3, ILT4, an agonist or antibody that binds Toll ligand receptor and a ligand that specifically binds with B7-H3.

A “costimulatory signal” refers to a signal, which in combination with a primary signal, such as TCR/CD3 ligation, leads to T cell proliferation and/or upregulation or downregulation of a molecule.

A bi-specific antibody molecule as employed herein refers to a molecule with two antigen binding domains, which may bind the same or different antigens. A BiTE is a subclass of bispecific antibody molecules. “Non-IgG-like” antibodies include antibodies that lack an Fc portion such as bispecific T cell engagers (BiTE), DART, tetravalent antiparallel structures (TandAbs) and VH-only bi-nanobodies. The non-IgG-like antibodies due to their lack of an Fc portion are smaller and have shorter in vivo half-lives.

As used herein, “Bi-specific T-cell engager” or “BiTE” or “BiTE molecule” are a class of bi-specific antibodies that direct T-cells to cancer cells. An exemplary Bi-specific T-cell engager antibodies comprises two antigen binding domains, for example, one of which targets a cancer antigen and the other of which targets an antigen on a T cell. A BiTE molecule usually targets one tumor antigen and one CD3 molecule simultaneously. By binding both T cells and cancer cells expressing cancer antigens, the bi-specific T-cell engager antibody mimics physiological processes observed during T cell targeting of cancer cells, and brings the T cells of the subject into proximity with the cancer cells, facilitating T cell cytotoxic activity and killing of cancer cells.

“Nucleic acid,” “polynucleotide,” and “oligonucleotide,” are used interchangeably in the present application. These terms refer only to the primary structure of the molecule. Thus, these terms include double- and single-stranded DNA, as well as double- and single-stranded RNA (e.g., messenger RNA (mRNA) or plasmid DNA (pDNA)). The terms “nucleic acid,” “polynucleotide,” and “oligonucleotide,” as used herein, are defined as it is generally understood by the skilled person as a molecule comprising two or more covalently linked nucleosides. Such covalently bound nucleosides can also be referred to as nucleic acid molecules or oligomers. Polynucleotides can be made recombinantly, enzymatically, or synthetically, e.g., by solid-phase chemical synthesis followed by purification. When referring to a sequence of the polynucleotide or nucleic acid, reference is made to the sequence or order of nucleobase moieties, or modifications thereof, of the covalently linked nucleotides or nucleosides. By “isolated” nucleic acid or polynucleotide is intended a nucleic acid molecule, DNA or RNA, which has been removed from its native environment. An isolated polynucleotide include recombinant polynucleotides maintained in heterologous host cells or purified (partially or substantially) polynucleotides in solution. Isolated RNA molecules include in vivo or in vitro RNA transcripts of polynucleotides. Isolated polynucleotides or nucleic acids further include such molecules produced synthetically. In addition, polynucleotides or a nucleic acid can be or can include a regulatory element such as a promoter, ribosome binding site, or a transcription terminator. Nucleic acids may be comprised in a vector. The term “vector” as used herein includes any vectors known to the skilled person including plasmid vectors, cosmid vectors, phage vectors such as lambda phage, viral vectors such as retroviral, adenoviral or baculoviral vectors, or artificial chromosome vectors such as bacterial artificial chromosomes (BAC), yeast artificial chromosomes (YAC), or P1 artificial chromosomes (PAC). Said vectors include expression as well as cloning vectors.

As used herein, the terms “peptide”, “polypeptide” and “protein” are used interchangeably and refer to a compound composed of amino acid residues covalently linked by peptide bonds. A protein or peptide must contain at least two amino acids, and there is no limit to the maximum number of amino acids that can constitute a protein or peptide sequence. Polypeptides include any peptide or protein comprising two or more amino acids connected to each other by peptide bonds. As used herein, the term refers to both short and long chains. Short chains are also commonly referred to in the art as peptides, oligopeptides, and oligomers, and long chains are commonly referred to as proteins in the art, and there are many types. “Polypeptide” includes, for example, biologically active fragments, substantially homologous polypeptides, oligopeptides, homodimers, heterodimers, polypeptide variants, modified polypeptides, derivatives, analogs, fusion proteins, and the like. Polypeptides include natural peptides, recombinant peptides or a combination thereof.

As used herein, the term “promoter/regulatory sequence” refers to a nucleic acid sequence required to express a gene product operably linked to a promoter/regulatory sequence. The term “constitutive” promoter refers to a nucleotide sequence that, when operably linked to a polynucleotide encoding or specifying a gene product, results in the production of a gene product in the cell under most or all physiological conditions of the cell. The term “inducible” promoter means that when operably linked to a polynucleotide encoding a specified gene product, it basically results in the production of a gene in the cell only when the inducer corresponding to the promoter is present in the cell The nucleotide sequence of the product.

As used herein, the term “expression” refers to a process by which a gene produces a biochemical, for example, a polypeptide. The process includes any manifestation of the functional presence of the gene within the cell including, without limitation, gene knockdown as well as both transient expression and stable expression. It includes without limitation transcription of the gene into messenger RNA (mRNA), and the translation of such mRNA into polypeptide(s). Expression of a gene produces a “gene product.”

As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

As used herein, the term “expression vector” refers to a vector comprising a recombinant polynucleotide comprising an expression control sequence operably linked to the nucleotide sequence to be expressed. The expression vector contains sufficient cis-acting elements for expression; other elements for expression can be provided by the host cell or in an in vitro expression system. Expression vectors include expression vectors known in the art, including cosmids, plasmids (for example, naked or contained in liposomes), and viruses incorporating recombinant polynucleotides (for example, lentivirus, retrovirus, adenovirus, and adeno-associated virus).

As used herein, the term “operably linked” or “transcription control” refers to a functional linkage between a regulatory sequence and a heterologous nucleic acid sequence, which results in the expression of the latter. For example, when the first nucleic acid sequence and the second nucleic acid sequence are arranged in a functional relationship, the first nucleic acid sequence and the second nucleic acid sequence are operably linked. For example, if a promoter affects the transcription or expression of a coding sequence, the promoter is operably linked to the coding sequence. The operably linked DNA sequences may be adjacent to each other, and for example, in the case where two protein coding regions need to be linked, the DNA sequences are in the same reading frame.

As used herein, the term “transfer vector” refers to a composition containing an isolated nucleic acid and a substance that can be used to deliver the isolated nucleic acid to the inside of a cell. Many vectors are known in the art, including but not limited to linear polynucleotides, polynucleotides associated with ionic or amphiphilic compounds, plasmids, and viruses. Therefore, a transfer vector can include autonomously replicating plasmids or viruses. The term transfer vector should also be interpreted to further include non-plasmid and non-viral compounds that facilitate the transfer of nucleic acids into cells, such as polylysine compounds, liposomes, and the like. Examples of virus transfer vectors include, but are not limited to, adenoviral vectors, adeno-associated virus vectors, retroviral vectors, lentiviral vectors, and the like.

As used herein, the term “host cell” can be any type of cell, e.g., a primary cell, a cell in culture, or a cell from a cell line. In specific aspects, the term “host cell” refers to a cell transfected with a nucleic acid molecule and the progeny or potential progeny of such a cell. Progeny of such a cell may not be identical to the parent cell transfected with the nucleic acid molecule, e.g., due to mutations or environmental influences that may occur in succeeding generations or integration of the nucleic acid molecule into the host cell genome.

“Percent (%) amino acid sequence identity” with respect to a polypeptide sequence as set forth herein is defined as the percentage of amino acid residues in a candidate sequence of interest to be compared that are identical with the amino acid residues in a particular polypeptide sequence as set forth herein (e.g. a particular polypeptide sequence characterized by a sequence identifier in the sequence listings), after aligning the sequences and introducing gaps, if necessary, to achieve the maximum percent sequence identity, and not considering any conservative substitutions as part of the sequence identity. A sequence alignment performed for determining percent amino acid sequence identity can be carried out according to procedures known in the art, as described for example in EP 1 241 179 B1, which is incorporated herewith by reference, including in particular page 9, line 35 to page 10, line 40 with the definitions used therein and Table 1 regarding possible conservative substitutions. For example, a skilled person can use publicly available computer software. Computer program methods for determining sequence identity include, but are not limited to BLAST, BLAST-2, ALIGN or Megalign (DNASTAR) software. According to one embodiment, the software alignment program used can be BLAST. A skilled person can determine appropriate parameters for measuring alignment, including any algorithms needed to achieve maximal alignment over the full length of the sequences subjected to comparison. According to one embodiment, the % identity values can be generated using the WU-BLAST-2 computer program (Altschul et al., 1996, Methods in Enzymology 266:460-480, which is incorporated herewith by reference). According to one embodiment, the following parameters are used, when carrying out the WU-BLAST-2 computer program: Most of the WU-BLAST-2 search parameters are set to the default values. The adjustable parameters were set with the following values: overlap span=1, overlap fraction=0.125, word threshold (T)=11, and scoring matrix=BLOSUM62. The HSP S and HSP S2 parameters, which are dynamic values used by BLAST-2, are established by the program itself depending upon the composition of the sequence of interest and composition of the database against which the sequence is being searched. However, the values can be adjusted to increase sensitivity. A % sequence identity value can be determined by dividing (a) the number of matching identical amino acid residues between a particular amino acid sequence as set forth herein which is subjected to comparison (e.g. a particular polypeptide sequence characterized by a sequence identifier in the sequence listings) and the candidate amino acid sequence of interest to be compared, for example the number of matching identical amino acid residues as determined by WU-BLAST-2, by (b) the total number of amino acid residues of the polypeptide sequence as set forth herein which is subjected to comparison (e.g. a particular polypeptide sequence characterized by a SEQ. ID. NO. in the sequence listings).

“Percent (%) nucleic acid sequence identity” with respect to a nucleic acid sequence as set forth herein is defined as the percentage of nucleotides in a candidate sequence of interest to be compared that are identical with the nucleotides in a particular nucleic acid sequence as set forth herein (e.g. a particular polypeptide sequence characterized by a sequence identifier in the sequence listings), after aligning the sequences and introducing gaps, if necessary, to achieve the maximum percent sequence identity. An alignment for purposes of determining percent nucleic acid sequence identity can be carried out according to procedures known in the art, as described for example in EP 1 241 179 B1. For example, a skilled person can use publicly available computer software, such as using publicly available computer software such as BLAST, BLAST-2, ALIGN or Megalign (DNASTAR) software. A skilled person can determine appropriate parameters for measuring alignment, including any algorithms needed to achieve maximal alignment over the full length of the sequences subjected to comparison. According to a preferred embodiment, the % identity values can be generated using the WU-BLAST-2 computer program. According to a preferred embodiment, the following computer program and parameters are used: The identity values used herein are generated by the BLASTN module of WU-BLAST-2 set to the default parameters, with overlap span and overlap fraction set to 1 and 0.125, respectively. A % nucleic acid sequence identity value can be obtained by dividing (a) the number of matching identical nucleotides between a particular nucleic acid sequence as set forth herein which is subjected to comparison (e.g. a particular nucleic acid sequence characterized by a sequence identifier in the sequence listings), and the comparison nucleic acid molecule of interest to be compared, for example the number of matching identical nucleotides as determined by WU-BLAST-2, by (b) the total number of nucleotide residues of the particular nucleic acid sequence as set forth herein which is subjected to comparison (e.g. a particular nucleic acid sequence characterized by a sequence identifier in the sequence listings).

As used herein, the term “homology” or “identity” refers to the identity of subunit sequence between two polymer molecules, for example, between two nucleic acid molecules, such as two DNA molecules or two RNA molecules, or between two polypeptide molecules. When subunit positions in two molecules are occupied by the same monomer subunit; for example, if the position of each of two DNA molecules is occupied by adenine, they are homologous or identical at that position. The homology between two sequences is a direct function of the number of matching or homologous positions; for example, if half of the positions in the two sequences (for example, 5 positions in a polymer of 10 subunits in length) are homologous, the two sequences are 50% homologous; if 90% of the positions (for example, 9 out of 10) are matched or homologous, then the two sequences are 90% homologous.

In the context of two or more nucleic acid or polypeptide sequences, identity percent refers to two or more sequences that are the same. When comparing and aligning for maximum correspondence in a comparison window or a designated area, as measured by using one of the following sequence comparison algorithms or by manual alignment and visual inspection, if the two sequences have a specified percentage of identical amino acid residues or nucleotides (e.g., 60% identity, optionally 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity over a specified region, or if not specified, over the entire sequence), then the two sequences are “substantially the same”. Optionally, the identity exists over a region of at least about 50 nucleotides (or 10 amino acids) in length, or more preferably over a region of 100 to 500 or 1000 or more nucleotides in length (Or 20, 50, 200 or more amino acids). For sequence comparison, usually a sequence serves as a reference sequence against which the test sequence is compared. When a sequence comparison algorithm is used, a test sequence and a reference sequence are input into a computer, and the sub-sequence coordinates and the sequence algorithm program parameters are specified, if necessary. Default program parameters can be used, or alternative parameters can be specified. Subsequently, the sequence comparison algorithm calculates the percent sequence identity of the test sequence relative to the reference sequence based on the program parameters. Methods of sequence alignment for comparison are well known in the art as disclosed above.

“Coding sequence” or a sequence “encoding” a particular molecule (e.g., a therapeutic molecule) is a nucleic acid that is transcribed (in the case of DNA) or translated (in the case of mRNA) into polypeptide, in vitro or in vivo, when operably linked to an appropriate regulatory sequence, such as a promoter. The boundaries of the coding sequence are determined by a start codon at the 5′ (amino) terminus and a translation stop codon at the 3′ (carboxy) terminus. Although a “stop codon” (TAG, TGA, or TAA) is not translated into an amino acid, it can be considered to be part of a coding region, but any flanking sequences, for example promoters, ribosome binding sites, transcriptional terminators, introns, and the like, are not part of a coding region.

A coding sequence can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the coding sequence.

As used herein, the term “recombinant DNA/RNA technology” refers to the manipulation of nucleic acid sequences outside of an organism. This technology comprises, but is not limited to, combining nucleic acid sequences (e.g., coding sequences, regulatory elements (e.g., promoters, enhancers, silencers, termination sequences), linkers (e.g., spacers, internal ribosome entry sites, cleavage sites) derived from a variety of sources, inserting nucleic acid sequences from a variety of sources in appropriate vectors (e.g., delivery vectors, expression vectors, integrating vectors), modifying or altering nucleotide sequences (e.g., by mutagenesis, insertion of modified nucleotides, 5′-capping, polyadenylation), synthesizing artificial nucleotide sequence. A variety of techniques well-known in the art (e.g., molecular cloning, polymerase chain reaction (PCR), digestion with restriction enzymes, in vitro ligation, mutagenesis, site-directed mutagenesis, prokaryotic and eukaryotic cell transformation or transduction, in vitro DNA/RNA synthesis, in vitro RNA-5′-capping, in vitro RNA-polyadenylation, complementary DNA (cDNA) synthesis, nucleic acid isolation, and the like) can be used to manipulate nucleic acid sequences outside an organism (see for example Green & Sambrook Molecular Cloning: A Laboratory Manual, volumes 1-3, 4th edition).

As used herein, the term “recombinant”, refers to any nucleic acid (e.g., DNA, or RNA), peptide (e.g., oligopeptide, polypeptide, or protein), cell, or organism, which is made by combining genetic material from two or more different sources. In some aspects, the recombinant nucleic acid, peptide, cell or organism comprises a portion of the genetic material from at least one source. In some aspects, “recombinant DNA” molecules can include DNA molecules derived from one organism and inserted in a host organism to produce new genetic combinations. In some aspects, “recombinant RNA” molecule (e.g., recombinant mRNA molecules) can include RNA molecules derived from one organism and inserted in a host organism to produce the expression of a desired genetic product in the host organism. In some aspects, “recombinant peptide” molecules can include amino acid molecules derived from an organism or cell, which are expressed from recombinant nucleic acid molecules.

As used herein, the term “isolated” means changed or removed from the natural state. For example, a nucleic acid or peptide naturally present in a living animal is not “isolated”, but the same nucleic acid or peptide that is partially or completely separated from a substance co-existing in its natural state is “isolated.” The isolated nucleic acid or protein may exist in a substantially purified form or may exist in a non-natural environment such as a host cell.

As used herein, the term “antigen” or “Ag” refers to a molecule that causes an immune response. The immune response can involve the production of antibodies or the activation of cells with specific immunity, or both. A skilled person in the art should understand that macromolecules including proteins or peptides can serve as an antigen. In addition, an antigen can be derived from recombinant or genomic DNA. When the term is used herein, the term includes any DNA including a nucleotide sequence or part of a nucleotide sequence encoding a protein that causes an immune response, and therefore encoding an “antigen”. In addition, the antigen need not be encoded only by the full-length nucleotide sequence of the gene. It is obvious that the present invention includes, but is not limited to, the use of partial nucleotide sequences of more than one gene, and these nucleotide sequences are arranged in different combinations to encode polypeptides eliciting a desired immune response. Moreover, the antigen does not need to be encoded by a “gene” at all. The antigen can be produced synthetically, or it can be derived from a biological sample, or it can be a macromolecule other than a polypeptide. Such biological samples may include, but are not limited to, tissue samples, tumor samples, cells or fluids with other biological components.

The term “antigen recognition unit” as used herein can include any molecular structure containing a polypeptide chain that has a specific shape that matches the epitope and recognizes the epitope, in which one or more non-covalent binding interactions stabilize the complex between the molecular structure and the epitope.

If the antigen recognition unit binds to an antigen with greater affinity or avidity compared with binding with other reference antigens (including polypeptides or other substances), the antigen recognition unit “specifically binds” to the antigen or is “immunoreactive with the antigen”.

As used herein, the term “tumor antigen” refers to an antigen common to specific hyperproliferative diseases. In certain aspects, the hyperproliferative disorder antigens of the disclosure are derived from cancer, e.g., NSCLC. In certain aspects, the tumor antigen of the present disclosure includes an epidermal growth factor receptor (EGFR) comprising a T790M mutation on human leukocyte antigen-A (HLA-A)*02:01.

The term “tumor” as used herein refers to any mass of tissue that results from excessive cell growth or proliferation, either benign (non-cancerous) or malignant (cancerous), including pre-cancerous lesions.

The term “primary tumor”, as used herein, refers to the original, or first, tumor formed in the subject's body.

The term “metastasis”, “metastatic”, “secondary tumor”, or “metastatic tumor”, as used herein, refer to cancer (e.g., a tumor) formed by cancer cells derived from a primary cancer (e.g., tumor) that spread to further locations or areas of the body.

As used herein, the term “specifically binds” refers to an antigen binding molecule that recognizes and binds a protein of a binding partner (such as a tumor antigen) present in a sample, but the antigen binding molecule does not substantially recognize or bind to other molecules in the sample.

As used herein, the terms “variable region” or “variable domain” are used interchangeably and are common in the art. The variable region typically refers to a portion of an immunoglobulin molecule (e.g. a portion of a light or heavy chain) or a portion of a T Cell Receptor (e.g. a portion of the alpha or beta chain), which differ in sequence among immunoglobulin or T Cell receptor molecules and are used in the binding and specificity of a particular molecule for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that CDRs of the light and heavy chains, or alpha and beta chains, are primarily responsible for the interaction and specificity of the immunoglobulin or T Cell receptor molecule with antigen.

The “hypervariable regions” in each chain are held together in close proximity by FRs, and with the hypervariable regions from the other chain, contribute to the formation of the antigen-binding site of immunoglobulin molecule (see Kabat et al, Sequences of Proteins of Immunological Interest, 1992). The hypervariable region generally comprises amino acid residues from a “complementarity determining region” or “CDR”, the latter being of highest sequence variability and/or involved in antigen recognition. A number of CDR definitions are in use and are encompassed herein. The Kabat definition is based on sequence variability and is the most commonly used (Kabat E A et al., supra). Chothia refers instead to the location of the structural loops (Chothia C & LeskAM (1987) J. Mol. Biol. 196: 901-917). The AbM definition is a compromise between the Kabat and the Chothia definitions and is used by Oxford Molecular's AbM antibody modelling software (Martin A C R et al., (1989) Proc. Natl Acad. Sci. USA, 86: 9268-72; Martin A C R et al., (1991) Methods Enzymol. 203: 121-153; Pedersen J T et al., (1992) Immunomethods, 1: 126-136; Rees A R et al., (1996) In Sternberg M. J. E. (ed.), Protein Structure Prediction. Oxford University Press, Oxford, 141-172). The contact definition has been recently introduced (Maccallum R M et al., (1996) J. Mol. Biol. 262: 732-7 45) and is based on an analysis of the complex structures available in the Protein Databank. The definition of the CDR by IMGT©, the international ImMunoGeneTics information System© (www.imgt.org) is based on the IMGT numbering for all immunoglobulin and T cell receptor V-REGIONs of all species (IMGT©, the international ImMunoGeneTics information System©; Lefranc M P et al., (1991) Nucleic Acids Res. 27(1): 209-12; Ruiz M et al., (2000) Nucleic Acids Res. 28(1): 219-21; Lefranc M P (2001) Nucleic Acids Res. 29(1): 207-9; Lefranc M P (2003) Nucleic Acids Res. 31(1): 307-10; Lefranc M P et al., (2005) Dev. Comp. Immunol. 29(3): 185-203; Kaas Q et al., (2007) Briefings in Functional Genomics & Proteomics, 6(4): 253-64).

In some aspects, the Complementarity Determining Regions (CDRs) disclosed herein are defined according to IMGT©. In some aspects, the CDRs are defined according to Chothia. In some aspects, the CDRs are defined according to Kabat.

In some aspects, alternate CDR sequences are provided for the same framework amino acid sequences of a variable region. In some aspects, the alternate CDR sequences are generated using software programs. In some aspects, different software can be used to generate alternate CDR sequences for the framework sequences of a variable region with different CDR sequences resulting from the use of the different software programs. In some aspects, the use of alternate CDR sequences can improve binding affinities of a bispecific antibody molecule to at least one antigen. In some aspects, alternate CDR sequences are used for affinity optimization of one or both antigen binding sites of a bispecific antibody molecule according to the present invention. In some aspects, the alternate CDRs are defined according to Kabat, Chothia, Paratome, AbM, Contact and/or IMGT annotations. In some aspects, the CDRs are defined according to more than one annotation.

As used herein, the terms “constant region” and “constant domain” are interchangeable and have their meaning common in the art. The constant region of an immunoglobulin molecule generally has a more conserved amino acid sequence relative to an immunoglobulin variable domain.

As used herein, an “epitope” is a term in the art and refers to a localized region of an antigen to which an engineered TCRs can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In certain aspects, the epitope to which an engineered TCRs binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization can be accomplished using any of the known methods in the art (e.g., Giege R et al, (1994) Acta Crystallogr D Biol Crystallogr 50(Pt 4): 339-350; McPherson A (1990) Eur J Biochem 189: 1-23; Chayen N E (1997) Structure 5: 1269-1274; McPherson A (1976) J Biol Chem 251: 6300-6303). Antigen crystals can be studied using well known X-ray diffraction techniques and can be refined using computer software such as X-PLOR (Yale University, 1992, distributed by Molecular Simulations, Inc.; see, e.g., Meth Enzymol (1985) volumes 114 & 115, eds Wyckoff H W et al.; U.S. 2004/0014194), and BUSTER (Bricogne G (1993) Acta Crystallogr D Biol Crystallogr 49(Pt 1): 37-60; Bricogne G (1997) Meth Enzymol 276A: 361-423, ed Carter C W; Roversi P et al, (2000) Acta Crystallogr D Biol Crystallogr 56(Pt 10): 1316-1323). Mutagenesis mapping studies can be accomplished using any method known to one of skill in the art. See, e.g., Champe M et al, (1995) J Biol Chem 270: 1388-1394 and Cunningham B C & Wells J A (1989) Science 244: 1081-1085 for a description of mutagenesis techniques, including alanine scanning mutagenesis techniques.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, chimeric antibodies, humanized antibodies, human antibodies, fusion proteins comprising an antibody, and any other modified immunoglobulin molecule so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

The term “antibody fragment” refers to a portion of an intact antibody. An “antigen-binding fragment,” “antigen-binding domain,” or “antigen-binding region,” refers to a portion of an intact antibody that binds to an antigen. An antigen-binding fragment can contain an antigen recognition site of an intact antibody (e.g., complementarity determining regions (CDRs) sufficient to bind antigen). Examples of antigen-binding fragments of antibodies include, but are not limited to Fab, Fab′, F(ab′)2, and Fv fragments, linear antibodies, and single chain antibodies. An antigen-binding fragment of an antibody can be derived from any animal species, such as rodents (e.g., mouse, rat, or hamster) and humans or can be artificially produced.

As used herein, the term “heavy chain” when used in reference to an antibody can refer to any distinct type, e.g., alpha (a), delta (d), epsilon (e), gamma (g), and mu (m), based on the amino acid sequence of the constant domain, which give rise to IgA, IgD, IgE, IgG, and IgM classes of antibodies, respectively, including subclasses of IgG, e.g., IgG1, IgG2, IgG3, and IgG4. Heavy chain amino acid sequences are well known in the art. In some aspects, the heavy chain is a human heavy chain.

As used herein, the term “light chain” when used in reference to an antibody can refer to any distinct type, e.g., kappa (K) or lambda (l) based on the amino acid sequence of the constant domains. Light chain amino acid sequences are well known in the art. In some aspects, the light chain is a human light chain.

The term “chimeric” antibodies or antigen-binding fragments thereof refers to antibodies or antigen-binding fragments thereof wherein the amino acid sequence is derived from two or more species. Typically, the variable region of both light and heavy chains corresponds to the variable region of antibodies or antigen-binding fragments thereof derived from one species of mammals (e.g. mouse, rat, rabbit, etc.) with the desired specificity, affinity, and capability while the constant regions are homologous to the sequences in antibodies or antigen-binding fragments thereof derived from another (usually human) to avoid eliciting an immune response in that species.

The term “humanized” antibody or antigen-binding fragment thereof refers to forms of non-human (e.g. murine) antibodies or antigen-binding fragments that are specific immunoglobulin chains, chimeric immunoglobulins, or fragments thereof that contain minimal non-human (e.g., murine) sequences. Typically, humanized antibodies or antigen-binding fragments thereof are human immunoglobulins in which residues from the complementary determining region (CDR) are replaced by residues from the CDR of a non-human species (e.g. mouse, rat, rabbit, hamster) that have the desired specificity, affinity, and capability (“CDR grafted”) (Jones et al., Nature 321:522-525 (1986); Riechmann et al., Nature 332:323-327 (1988); Verhoeyen et al., Science 239:1534-1536 (1988)). In some instances, the Fv framework region (FR) residues of a human immunoglobulin are replaced with the corresponding residues in an antibody or fragment from a non-human species that has the desired specificity, affinity, and capability. The humanized antibody or antigen-binding fragment thereof can be further modified by the substitution of additional residues either in the Fv framework region and/or within the non-human CDR residues to refine and optimize the specificity, affinity, and/or capability of the antibody or antigen-binding fragment thereof. In general, the humanized antibody or antigen-binding fragment thereof will comprise substantially all of at least one, and typically two or three, variable domains containing all or substantially all of the CDR regions that correspond to the non-human immunoglobulin whereas all or substantially all of the FR regions are those of a human immunoglobulin consensus sequence. The humanized antibody or antigen-binding fragment thereof can also comprise at least a portion of an immunoglobulin constant region or domain (Fc), typically that of a human immunoglobulin. Examples of methods used to generate humanized antibodies are described in U.S. Pat. No. 5,225,539; Roguska et al., Proc. Natl. Acad. Sci., USA, 91(3):969-973 (1994), and Roguska et al., Protein Eng. 9(10):895-904 (1996). In some aspects, a “humanized antibody” is a resurfaced antibody.

The term “human” antibody or antigen-binding fragment thereof means an antibody or antigen-binding fragment thereof having an amino acid sequence derived from a human immunoglobulin gene locus, where such antibody or antigen-binding fragment is made using any technique known in the art. This definition of a human antibody or antigen-binding fragment thereof includes intact or full-length antibodies and fragments thereof.

As used herein, the term “tumor heterogeneity” means that, after multiple divisions and proliferation during the growth of a tumor, daughter cells of the tumor its show molecular biological or genetic changes, so that there are differences in the growth rate, invasion ability, and drug sensitivity, prognosis and other aspects of the tumor. It is one of the characteristics of malignant tumors.

As used herein, the term “cancer” refers to a broad group of various diseases characterized by the uncontrolled growth of abnormal cells (e.g., malignant cells) in the body. Unregulated cell division and growth results in the formation of malignant tumors that invade neighboring tissues through local spread and can also metastasize to distant parts of the body through the lymphatic system or bloodstream. In some aspects, the methods of the present disclosure can be used to reduce the size of a primary tumor or a metastatic tumor, or treat a primary tumor or a metastatic tumor. The conditions that can be treated or prevented by the method of the present invention include, for example, various neoplasms, including benign or malignant tumors, various hyperplasias, and the like. The method of the present disclosure can achieve the inhibition and/or reversal of the undesirable hyperproliferative cell growth involved in such conditions. In some aspects, the cancer can be lung cancer, e.g., non-small cell lung cancer (NSCLC).

As used herein, the term “transfected” or “transformed” or “transduced” refers to the process by which exogenous nucleic acid is transferred or introduced into a host cell. A “transfected” or “transformed” or “transduced” cell is a cell that has been transfected, transformed or transduced with exogenous nucleic acid. The cells include primary cell of a subject and progenies thereof.

As used herein, “refractory” as used herein refers to a disease, such as cancer, which does not respond to treatment. In an embodiment, a refractory cancer may be resistant to treatment before or at the beginning of the treatment. In other embodiments, a refractory cancer may become resistant during treatment. Refractory cancers are also called resistant cancers. In the present disclosure, refractory cancers include, but are not limited to, cancers that are not sensitive to EGFR-TKIs. In some aspects, refractory or recurrent malignant tumors can use the treatment methods disclosed herein.

As used herein, “relapsed” as used herein refers to the return of the signs and symptoms of a disease (e.g. cancer) or the return of a disease such as cancer during a period of improvement, for example, after a therapy, such as a previous treatment of cancer therapy.

As used herein, the terms “treat,” “treated,” and “treating” mean both therapeutic and prophylactic treatment or preventative measures wherein the object is to reverse, alleviate, ameliorate, lessen, inhibit, slow down progression, development, severity or recurrence of an undesired symptom, complication, condition, biochemical indicia of a disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. In some aspects, treatment includes eliciting a clinically significant response without excessive levels of side effects. In some aspects, treatment includes prolonging survival as compared to expected survival if not receiving treatment. As used herein, the term “amelioration” or “ameliorating” refers to a lessening of severity of at least one indicator of a condition or disease. As used herein, the term “preventing” or “prevention” refers to delaying or forestalling the onset, development or progression of a condition or disease for a period of time, including weeks, months, or years. As used herein, the term “prophylactic” (e.g., “prophylactic agent”, “prophylactic treatment”, “prophylactically effective amount”), refers to any complete or partial prevention of a disease or symptom thereof and/or can be therapeutic in terms of a partial or complete cure for a disease and/or adverse effect and/or symptom attributable to the disease.

As used herein, the terms “individual” and “subject” have the same meaning herein, and can be a human and animal from other species. As used herein, the terms “subject” and “patient” are used interchangeably. The subject can be an animal. In some aspects, the subject is a mammal such as a non-human animal (e.g., cow, pig, horse, cat, dog, rat, mouse, monkey or other primate, etc.). In some aspects, the subject is a human. In some aspects, the patient is a subject who has a disease, disorder, or condition, or is at risk of suffering from a disease, disorder, or condition, or is otherwise in need of the compositions and methods provided herein.

As used herein, the terms “therapeutically effective amount”, “therapeutically effective”, “effective amount” or “in an effective amount” are used interchangeably herein and refer to the amount of a compound, preparation, substance or composition that is effective to achieve a specific biological result as described herein, such as but not limited to treating or reducing the growth of a cancer or tumor. When indicating “immunologically effective amount”, “anti-tumor effective amount”, “tumor-suppressing effective amount” or “therapeutically effective amount”, the precise number of immune effector cells and therapeutic agents of the present invention to be administered can be determined by a physician in consideration of the individual's age, weight, tumor size, degree of infection or metastasis, and the condition of a patient (subject). An effective amount of immune effector cells refers to, but is not limited to, the number of immune effector cells which can increase, enhance or prolong the anti-tumor activity of immune effector cells; increase the number of anti-tumor immune effector cells or activated immune effector cells; promote tumor regression, tumor shrinkage and/or tumor necrosis.

The term “pharmaceutically acceptable” as used herein refers to those compounds, materials, compositions, formulations, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “excipient” refers to any substance, not itself a therapeutic agent, which can be used in a composition for delivery of an active therapeutic agent to a subject or combined with an active therapeutic agent (e.g., to create a pharmaceutical composition) to improve its handling or storage properties or to permit or facilitate formation of a dose unit of the composition. Excipients include, but are not limited to, solvents, penetration enhancers, wetting agents, antioxidants, lubricants, emollients, substances added to improve appearance or texture of the composition and substances used to form hydrogels. Any such excipients can be used in any dosage forms according to the present disclosure. The foregoing classes of excipients are not meant to be exhaustive but merely illustrative as a person of ordinary skill in the art would recognize that additional types and combinations of excipients could be used to achieve the desired goals for delivery of a drug. The excipient can be an inert substance, an inactive substance, and/or a not medicinally active substance. The excipient can serve various purposes.

A person skilled in the art can select one or more excipients with respect to the particular desired properties by routine experimentation and without any undue burden. The amount of each excipient used can vary within ranges conventional in the art. Techniques and excipients which can be used to formulate dosage forms are described in Handbook of Pharmaceutical Excipients, 6th edition, Rowe et al., Eds., American Pharmaceuticals Association and the Pharmaceutical Press, publications department of the Royal Pharmaceutical Society of Great Britain (2009); and Remington: the Science and Practice of Pharmacy, 21st edition, Gennaro, Ed., Lippincott Williams & Wilkins (2005).

As used herein, the term “immune response” refers to a biological response within an organism against a foreign agent or abnormal cell (e.g., a tumor cell), wherein the response protects the organism against such agents/cells and diseases caused by them. An immune response is mediated by the action of a cell of the immune system (e.g., a T lymphocyte (T cell), B lymphocyte (B cell), natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the organism's body of invading pathogens, cells or tissues infected with pathogens, cancerous or other abnormal cells, or, in cases of autoimmunity or pathological inflammation, normal human cells or tissues. In some aspects, an immune reaction includes, e.g., activation or inhibition of a T cell, e.g., an effector T cell or a Th cell, such as a CD4+ or CD8+ T cell, or the inhibition of a regulatory T cell (Treg cell).

As used herein, the term “immune effector cell” refers to a cell that exerts an effector function during an immune response, including, for example, immune cells secreting cytokines and/or chemokines, killing microorganisms, secreting antibodies, and recognizing or eliminating tumor cells. In some embodiments, immune effector cells include T cells (cytotoxic T cells, helper T cells, tumor-infiltrating T cells), B cells, natural killer cells, neutrophils, macrophages, and dendritic cells.

As used herein, the term “immune effector function” includes any function mediated by the composition of the immune system, which can lead to inhibition of tumor growth and/or inhibition of tumorigenesis, including inhibition the spread and metastasis of a tumor. Preferably, the immune effector function kills tumor cells.

As used herein, the term “antigen presenting cell” or “APC” refers to a cell in the immune system that display a complex of foreign antigens and major histocompatibility complex (MHC) on the surface, such as helper cells (such as B cells, dendritic cells, etc.). T cells can recognize these complexes using a T cell receptor (TCR) thereof. APC processes the antigen and presents it to T cells.

As used herein, the term “immunotherapy” refers to the treatment of a disease by inducing, enhancing, suppressing or otherwise modifying an immune response.

As used herein, the term “autologous” refers to any material derived from an individual that will later be reintroduced into that same individual.

As used herein, the term “anti-tumor effect” refers to a biological effect that can be manifested in various ways, including but not limited to, for example, reduction in tumor volume, reduction in the number of tumor cells, reduction in the number of metastases, increase in life expectancy, reduction in tumor cell proliferation, and reduction in tumor cell survival rate, or improvement in various physiological symptoms related to cancerous conditions. The “anti-tumor effect” can also be expressed by the ability of the peptides, polynucleotides, cells and antibodies of the present disclosure to prevent or reduce the frequency of tumorigenesis.

The term “chemotherapy” as used herein refers to a wide variety of chemotherapeutic agents that may be used in accordance with the present embodiments. The term “chemotherapy” refers to the use of drugs to treat cancer. A “chemotherapeutic agent” is used to connote a compound or composition that is administered in the treatment of cancer.

The term “pharmaceutical composition” refers to a preparation which is in such form as to permit the biological activity of the active ingredient to be effective, and which contains no additional components which are unacceptably toxic to a subject to which the composition would be administered. The composition can be sterile.

The terms “administer,” “administering,” “administration,” and the like, as used herein, refer to methods that can be used to enable delivery of a drug, e.g., an engineered TCRs disclosed herein, to the desired site(s) of biological action (e.g., intravenous administration). Administration techniques that can be employed with the agents and methods described herein are found in e.g., Goodman and Gilman, The Pharmacological Basis of Therapeutics, current edition, Pergamon; and Remington's, Pharmaceutical Sciences, current edition, Mack Publishing Co., Easton, Pa.

It is understood that wherever aspects are disclosed herein with the language “comprising,” otherwise analogous aspects disclosed in terms of “consisting of” and/or “consisting essentially of” are also provided.

### II. EGFR Epitope

Certain aspects of the disclosure relate to T-cell receptors that bind to a Epidermal growth factor receptor (EGFR) epitope comprising a T790M mutation on human leukocyte antigen-A (HLA-A)*02:01 MQLMPFGCLL (SEQ ID NO: 1). In some aspects, the disclosure relates to tumor antigen-specific peptides, such as to the EGFR peptides that include a mutant EGFR sequence (e.g., a peptide having an insertion, substitution or deletion relative to a wildlype EGR sequence).

The EGFR T790M mutation is the second most frequent EGFR mutation in NSCLC following the L858R mutation. The epitope is endogenously processed and presented on tumor cells by HLA-A2 complexes. In certain aspects, the tumor antigen-specific peptide (e.g., EGFR mutant peptide) is immunogenic or antigenic.

### III. Engineered TCRs

T-cell receptors (TCRs) are membrane bound surface receptors of T-cells that recognize antigenic peptides in the major histocompatibility complex (MHC) on the surface of antigen-presenting cells. TCR2 includes an α-chain and a β-chain that are both subdivided into a variable and a constant region. The variable regions of the TCR are in contact with the antigen and the MHC. In contrast to antibodies, TCRs recognize small and continuous peptide antigens that are presented to them via the MHC of antigen presenting cells. TCRs do not act directly against antigens but trigger a signaling cascade inside the T-cell for further actions.

In some aspects, provided herein are engineered TCRs (also referred to herein as recombinant TCRs) which specifically bind to an epitope of an epidermal growth factor receptor (EGFR) comprising a T790M mutation presented on a human leukocyte antigen-A (HLA-A)*02:01. In some aspects, the engineered TCR specifically binds an amino acid sequence comprising SEQ ID NO: 1 (Yamada, T., et al., PLOS 8(11):e78389, 2013).

In some aspects, the engineered TCR of the present disclosure comprises a target-specific binding element, which is also referred to as an antigen recognition portion. For example, the antigen recognition portion can be selected to recognize the target antigen as a cell surface marker associated with a specific disease state on the target cell. In some aspect, the antigen recognition portion specifically recognizes the T790M mutation of EGFR on human leukocyte antigen-A (HLA-A)*02:01.

In some aspects, the extracellular domain of engineered TCR of the disclosure can be derived from natural sources or from recombinant sources. In the case of natural sources, the domain can be derived from any protein, especially a membrane-bound or transmembrane protein. In one aspect, the extracellular domain can associate with the transmembrane domain. Extracellular domains are particularly useful in the present invention.

In some aspects, the linker of the present disclosure is optionally a short oligopeptide of 2 to 10 amino acids in length, or a polypeptide linker that can form a connection between the alpha and beta chains of an engineered TCR. In some aspects, the TCR alpha and TCR β chains are connected to one another by an amino acid sequence that comprises a furin protease recognition site. In some aspects, the linker is cleaved to separate the alpha and beta chains of the engineered TCR. In some aspects, a glycine-serine doublet can be a suitable linker.

In some aspects, the linker sequence comprises the amino acid sequence:

(SEQ ID NO: 22)
RAKRSGSGATNFSLLKQAGDVEENPGP.

In some aspects, the linker sequence comprises the nucleic acid sequence:

(SEQ ID NO: 23)
CGTGCCAAGCGAAGCGGATCTGGCGCCACGAACTTCTCTCTGTTA
AAGCAAGCAGGAGATGTTGAAGAAAACCCCGGGCCT

In some aspects, if the engineered TCR contains a CD3gamma, delta, or epsilon polypeptide, the cytoplasmic domain of the engineered TCR can include intracellular signaling domains; and TCRalpha and TCRbeta subunits usually lack signaling domains. The intracellular signaling domain is generally responsible for activating at least one normal effector function of immune cells into which the TCR has been introduced. In some aspects, the engineered TCR binds epidermal growth factor receptor (EGFR) comprising a T790M mutation on human leukocyte antigen-A (HLA-A)*02:01.

In some aspects, an engineered TCRs provided herein binds epidermal growth factor receptor (EGFR) comprising a T790M mutation on human leukocyte antigen-A (HLA-A)*02:01 and comprises six CDRs (e.g., a CDR1β, a CDR2β, a CDR3β, a CDR1α, a CDR2α, and a CDR3α). The CDRs can be determined by a number of algorithms in the art, such as IMGT, abYsis, and Kabat.

In some aspects, the genetically engineered antigen receptors include recombinant T cell receptors (TCRs) and/or TCRs cloned from naturally occurring T cells. In some aspects, the TCR molecule comprises a variable α and β chains (also known as TCRα and TCRβ, respectively) or a variable γ and δ chains (also known as TCRγ and TCRδ, respectively) and is capable of specifically binding to an antigen peptide bound to a MHC receptor. In some embodiments, the TCR is in the ap form. In some aspects, the TCR comprises signal peptide sequences.

The term “chimeric” TCR refers to TCRs wherein the amino acid sequence is derived from two or more species. Typically, the variable regions of the TCR correspond to the variable region of TCRs derived from one species of mammals (e.g. mouse, rat, rabbit, etc.) with the desired specificity, affinity, and capability while the constant regions are homologous to the sequences in TCRs derived from another (usually human) to avoid eliciting an immune response in that species. The term “humanized” TCR refers to forms of non-human (e.g. murine) TCRs that contain minimal non-human (e.g., murine) sequences. Typically, humanized TCRs are human TCRs in which residues from the complementarity determining regions (CDRs) are replaced by residues from the CDRs of a non-human species (e.g. mouse, rat, rabbit, hamster) that have the desired specificity, affinity, and capability (“CDR grafted”. Examples of methods used to generate humanized TCRs are described in (Chen et al., A humanized TCR retaining authentic specificity and affinity conferred potent anti-tumour cytotoxicity, Immunology, 2018, 155(1): 123-136).

In some aspects, the signal peptide sequence comprises the amino acid sequence:

(SEQ ID NO: 24)
MSIGLLCCVAFSLLWASPVNA,
or
(SEQ ID NO: 25)
MKSLRVLLVILWLQLSWVWS.

In some aspects, the signal peptide sequence comprises the nucleic acid sequence:

(SEQ ID NO: 26)
ATGAGCATCGGGCTCCTGTGCTGTGTGGCCTTTTCTCTCCTGTGG
GCAAGTCCAGTG,
or
(SEQ ID NO: 27)
ATGAAATCCTTGAGAGTTTTACTGGTGATCCTGTGGCTTCAGTTA
AGCTGGGTTTGGAGCCAA.

Typically, TCRs that exist in ap and 76 forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules. In some embodiments, a TCR also can contain a constant domain, a transmembrane domain and/or a short cytoplasmic tail (see, e.g., Janeway et al, Immunobiology: The Immune System in Health and Disease, 3rd Ed., Current Biology Publications, p. 433, 1997). For example, in some aspects, each chain of the TCR can possess one N-terminal immunoglobulin variable domain, one immunoglobulin constant domain, a transmembrane region, and a short cytoplasmic tail at the C-terminal end. In some aspects, a TCR is associated with invariant proteins of the CD3 complex involved in mediating signal transduction.

In some aspects, a TCR includes any TCR or functional fragment, such as an antigen-binding portion of a TCR that binds to a specific antigenic peptide bound in an MHC molecule, i.e. MHC-peptide complex. In some aspects, the antigen-binding portion or antigen-binding fragment of a TCR, refers to a molecule that contains a portion of the structural domains of a TCR, but that binds the antigen (e.g. MHC-peptide complex) to which the full TCR binds. In some cases, an antigen-binding portion contains the variable domains of a TCR, such as variable a chain and variable β chain of a TCR, sufficient to form a binding site for binding to a specific MHC-peptide complex, such as generally where each chain contains three complementarity determining regions.

In some aspects, the polypeptides that are encoded by the polynucleotides described within this disclosure, comprise a TCR alpha chain and a TCR β chain, wherein (i) the two chains are present in a physical association with one another (e.g., in a complex) and are non-covalently joined to one another (e.g., by a peptide bond), or (ii) the two chains are covalently joined to one another, such as by a disulfide or other covalent linkage that is not a peptide bond. Other suitable linkages can comprise, for example, substituted or unsubstituted polyalkylene glycol, and combinations of ethylene glycol and propylene glycol in the form of, for example, copolymers. In some aspects, the two polypeptides that constitute the TCR alpha chain and a TCR β chain can both be included in a single polypeptide, such as a fusion protein. In some aspects, the two polypeptides that constitute the TCR alpha chain and a TCR β chain can be separate, i.e., not linked.

In some aspects, the variable domains of the TCR chains associate to form loops, or complementarity determining regions (CDRs) analogous to immunoglobulins, which confer antigen recognition and determine peptide specificity by forming the binding site of the TCR molecule and determine peptide specificity.

In some aspects, the TCR comprises any of the sequences disclosed in Table 1, or combinations thereof.

TABLE 1

| Description | Sequence |
|---|---|
| α CDR 1 | NSAFQY (SEQ ID NO: 2) |
| α CDR 2 | TYSSGN (SEQ ID NO: 3) |
| α CDR 3 | CAMNRDDKIIF (SEQ ID NO: 4) |
| β CDR 1 | MNHNS (SEQ ID NO: 5) |
| β CDR 2 | SASEGT (SEQ ID NO: 6) |
| β CDR 3 | ASSVSPGNYGYT (SEQ ID NO: 7) |
| α CDR 1 | AACAGTGCTTTTCAATAC (SEQ ID NO: 8) |
| α CDR 2 | ACATACTCCAGTGGTAAC (SEQ ID NO: 9) |
| α CDR 3 | GCAATGAATCGGGATGACAAGATCATC (SEQ ID NO: 10) |
| β CDR 1 | ATGAACCATAACTCC (SEQ ID NO: 11) |
| β CDR 2 | TCAGCTTCTGAGGGTACC (SEQ ID NO: 12) |
| β CDR 3 | GCCAGCAGTGTTTCCCCTGGAAACTATGGCTACACC |
| | (SEQ ID NO: 13) |
| Full TCR | MSIGLLCCVAFSLLWASPVNAGVTQTPKFQVLKTGQSMTL |
| Sequence | QCAQDMNHNSMYWYRQDPGMGLRLIYYSASEGTTDKGE |
| | VPNGYNVSRLNKREFSLRLESAAPSQTSVYFCASSVSPGNY |
| | GYTFGSGTRLTVVEDLNKVFPPEVAVFEPSEAEISHTQKAT |
| | LVCLATGFFPDHVELSWWVNGKEVHSGVCTDPQPLKEQP |
| | ALNDSRYCLSSRLRVSATFWQNPRNHFRCQVQFYGLSEND |
| | EWTQDRAKPVTQIVSAEAWGRADCGFTSVSYQQGVLSATI |
| | LYEILLGKATLYAVLVSALVLMAMVKRKDFRAKRSGSGA |
| | TNFSLLKQAGDVEENPGPMKSLRVLLVILWLQLSWVWSQ |
| | QKEVEQDPGPLSVPEGAIVSLNCTYSNSAFQYFMWYRQYS |
| | RKGPELLMYTYSSGNKEDGRFTAQVDKSSKYISLFIRDSQP |
| | SDSATYLCAMNRDDKIIFGKGTRLHILPNIQNPDPAVYQLR |
| | DSKSSDKSVCLFTDFDSQTNVSQSKDSDVYITDKCVLDMRS |
| | MDFKSNSAVAWSNKSDFACANAFNNSIIPEDTFFPSPESSCD |
| | VKLVEKSFETDTNLNFQNLSVIGFRILLLKVAGFNLLMTLR |
| | LWSS (SEQ ID NO: 28) |
| Full TCR | ATGAGCATCGGGCTCCTGTGCTGTGTGGCCTTTTCTCTCC |
| Sequence | TGTGGGCAAGTCCAGTGAATGCTGGTGTCACTCAGACCC |
| | CAAAATTCCAGGTCCTGAAGACAGGACAGAGCATGACA |
| | CTGCAGTGTGCCCAGGATATGAACCATAACTCCATGTAC |
| | TGGTATCGACAAGACCCAGGCATGGGACTGAGGCTGATT |
| | TATTACTCAGCTTCTGAGGGTACCACTGACAAAGGAGAA |
| | GTCCCCAATGGCTACAATGTCTCCAGATTAAACAAACGG |
| | GAGTTCTCGCTCAGGCTGGAGTCGGCTGCTCCCTCCCAG |
| | ACATCTGTGTACTTCTGTGCCAGCAGTGTTTCCCCTGGAA |
| | ACTATGGCTACACCTTCGGTTCGGGGACCAGGTTAACCG |
| | TTGTAGAGGACCTGAACAAGGTGTTCCCACCCGAGGTCG |
| | CTGTGTTTGAGCCATCAGAAGCAGAGATCTCCCACACCC |
| | AAAAGGCCACACTGGTGTGCCTGGCCACAGGCTTCTTCC |
| | CTGACCACGTGGAGCTGAGCTGGTGGGTGAATGGGAAG |
| | GAGGTGCACAGTGGGGTCTGCACGGACCCGCAGCCCCTC |
| | AAGGAGCAGCCCGCCCTCAATGACTCCAGATACTGCCTG |
| | AGCAGCCGCCTGAGGGTCTCGGCCACCTTCTGGCAGAAC |
| | CCCCGCAACCACTTCCGCTGTCAAGTCCAGTTCTACGGG |
| | CTCTCGGAGAATGACGAGTGGACCCAGGATAGGGCCAA |
| | ACCCGTCACCCAGATCGTCAGCGCCGAGGCCTGGGGTAG |
| | AGCAGACTGTGGCTTTACCTCGGTGTCCTACCAGCAAGG |
| | GGTCCTGTCTGCCACCATCCTCTATGAGATCCTGCTAGG |
| | GAAGGCCACCCTGTATGCTGTGCTGGTCAGCGCCCTTGT |
| | GTTGATGGCCATGGTCAAGAGAAAGGATTTCCGTGCCAA |
| | GCGAAGCGGATCTGGCGCCACGAACTTCTCTCTGTTAAA |
| | GCAAGCAGGAGATGTTGAAGAAAACCCCGGGCCTATGA |
| | AATCCTTGAGAGTTTTACTGGTGATCCTGTGGCTTCAGTT |
| | AAGCTGGGTTTGGAGCCAACAGAAGGAGGTGGAGCAGG |
| | ATCCTGGACCACTCAGTGTTCCAGAGGGAGCCATTGTTT |
| | CTCTCAACTGCACTTACAGCAACAGTGCTTTTCAATACTT |
| | CATGTGGTACAGACAGTATTCCAGAAAAGGCCCTGAGTT |
| | GCTGATGTACACATACTCCAGTGGTAACAAAGAAGATG |
| | GAAGGTTTACAGCACAGGTCGATAAATCCAGCAAGTAT |
| | ATCTCCTTGTTCATCAGAGACTCACAGCCCAGTGATTCA |
| | GCCACCTACCTCTGTGCAATGAATCGGGATGACAAGATC |
| | ATCTTTGGAAAAGGGACACGACTTCATATTCTCCCCAAT |
| | ATCCAGAACCCTGACCCTGCCGTGTACCAGCTGAGAGAC |
| | TCTAAATCCAGTGACAAGTCTGTCTGCCTATTCACCGATT |
| | TTGATTCTCAAACAAATGTGTCACAAAGTAAGGATTCTG |
| | ATGTGTATATCACAGACAAATGCGTGCTAGACATGAGGT |
| | CTATGGACTTCAAGAGCAACAGTGCTGTGGCCTGGAGCA |
| | ACAAATCTGACTTTGCATGTGCAAACGCCTTCAACAACA |
| | GCATTATTCCAGAAGACACCTTCTTCCCCAGCCCAGAAA |
| | GTTCCTGTGATGTCAAGCTGGTCGAGAAAAGCTTTGAAA |
| | CAGATACGAACCTAAACTTTCAAAACCTGTCAGTGATTG |
| | GGTTCCGAATCCTCCTCCTGAAAGTGGCCGGGTTTAATC |
| | TGCTCATGACGCTGCGGCTGTGGTCCAGCTGA |
| | (SEQ ID NO: 29) |
| α Chain | MKSLRVLLVILWLQLSWVWSQQKEVEQDPGPLSVPEGAIV |
| Amino Acid | SLNCTYSNSAFQYFMWYRQYSRKGPELLMYTYSSGNKED |
| | GRFTAQVDKSSKYISLFIRDSQPSDSATYLCAMNRDDKIIFG |
| | KGTRLHILPNIQNPDPAVYQLRDSKSSDKSVCLFTDFDSQT |
| | NVSQSKDSDVYITDKCVLDMRSMDFKSNSAVAWSNKSDF |
| | ACANAFNNSIIPEDTFFPSPESSCDVKLVEKSFETDTNLNFQ |
| | NLSVIGFRILLLKVAGFNLLMTLRLWSS (SEQ ID NO: 30) |
| α Chain | ATGAAATCCTTGAGAGTTTTACTGGTGATCCTGTGGCTTC |
| Nucleotide | AGTTAAGCTGGGTTTGGAGCCAACAGAAGGAGGTGGAG |
| Sequence | CAGGATCCTGGACCACTCAGTGTTCCAGAGGGAGCCATT |
| | GTTTCTCTCAACTGCACTTACAGCAACAGTGCTTTTCAAT |
| | ACTTCATGTGGTACAGACAGTATTCCAGAAAAGGCCCTG |
| | AGTTGCTGATGTACACATACTCCAGTGGTAACAAAGAAG |
| | ATGGAAGGTTTACAGCACAGGTCGATAAATCCAGCAAG |
| | TATATCTCCTTGTTCATCAGAGACTCACAGCCCAGTGATT |
| | CAGCCACCTACCTCTGTGCAATGAATCGGGATGACAAGA |
| | TCATCTTTGGAAAAGGGACACGACTTCATATTCTCCCCA |
| | ATATCCAGAACCCTGACCCTGCCGTGTACCAGCTGAGAG |
| | ACTCTAAATCCAGTGACAAGTCTGTCTGCCTATTCACCG |
| | ATTTTGATTCTCAAACAAATGTGTCACAAAGTAAGGATT |
| | CTGATGTGTATATCACAGACAAATGCGTGCTAGACATGA |
| | GGTCTATGGACTTCAAGAGCAACAGTGCTGTGGCCTGGA |
| | GCAACAAATCTGACTTTGCATGTGCAAACGCCTTCAACA |
| | ACAGCATTATTCCAGAAGACACCTTCTTCCCCAGCCCAG |
| | AAAGTTCCTGTGATGTCAAGCTGGTCGAGAAAAGCTTTG |
| | AAACAGATACGAACCTAAACTTTCAAAACCTGTCAGTGA |
| | TTGGGTTCCGAATCCTCCTCCTGAAAGTGGCCGGGTTTA |
| | ATCTGCTCATGACGCTGCGGCTGTGGTCCAGCTGA |
| | (SEQ ID NO: 31) |
| β Chain | MSIGLLCCVAFSLLWASPVNAGVTQTPKFQVLKTGQSMTL |
| Amino Acid | QCAQDMNHNSMYWYRQDPGMGLRLIYYSASEGTTDKGE |
| Sequence | VPNGYNVSRLNKREFSLRLESAAPSQTSVYFCASSVSPGNY |
| | GYTFGSGTRLTVVEDLNKVFPPEVAVFEPSEAEISHTQKAT |
| | LVCLATGFFPDHVELSWWVNGKEVHSGVCTDPQPLKEQP |
| | ALNDSRYCLSSRLRVSATFWQNPRNHFRCQVQFYGLSEND |
| | EWTQDRAKPVTQIVSAEAWGRADCGFTSVSYQQGVLSATI |
| | LYEILLGKATLYAVLVSALVLMAMVKRKDF |
| | (SEQ ID NO: 32) |
| β Chain | ATGAGCATCGGGCTCCTGTGCTGTGTGGCCTTTTCTCTCC |
| Nucleic Acid | TGTGGGCAAGTCCAGTGAATGCTGGTGTCACTCAGACCC |
| Sequence | CAAAATTCCAGGTCCTGAAGACAGGACAGAGCATGACA |
| | CTGCAGTGTGCCCAGGATATGAACCATAACTCCATGTAC |
| | TGGTATCGACAAGACCCAGGCATGGGACTGAGGCTGATT |
| | TATTACTCAGCTTCTGAGGGTACCACTGACAAAGGAGAA |
| | GTCCCCAATGGCTACAATGTCTCCAGATTAAACAAACGG |
| | GAGTTCTCGCTCAGGCTGGAGTCGGCTGCTCCCTCCCAG |
| | ACATCTGTGTACTTCTGTGCCAGCAGTGTTTCCCCTGGAA |
| | ACTATGGCTACACCTTCGGTTCGGGGACCAGGTTAACCG |
| | TTGTAGAGGACCTGAACAAGGTGTTCCCACCCGAGGTCG |
| | CTGTGTTTGAGCCATCAGAAGCAGAGATCTCCCACACCC |
| | AAAAGGCCACACTGGTGTGCCTGGCCACAGGCTTCTTCC |
| | CTGACCACGTGGAGCTGAGCTGGTGGGTGAATGGGAAG |
| | GAGGTGCACAGTGGGGTCTGCACGGACCCGCAGCCCCTC |
| | AAGGAGCAGCCCGCCCTCAATGACTCCAGATACTGCCTG |
| | AGCAGCCGCCTGAGGGTCTCGGCCACCTTCTGGCAGAAC |
| | CCCCGCAACCACTTCCGCTGTCAAGTCCAGTTCTACGGG |
| | CTCTCGGAGAATGACGAGTGGACCCAGGATAGGGCCAA |
| | ACCCGTCACCCAGATCGTCAGCGCCGAGGCCTGGGGTAG |
| | AGCAGACTGTGGCTTTACCTCGGTGTCCTACCAGCAAGG |
| | GGTCCTGTCTGCCACCATCCTCTATGAGATCCTGCTAGG |
| | GAAGGCCACCCTGTATGCTGTGCTGGTCAGCGCCCTTGT |
| | GTTGATGGCCATGGTCAAGAGAAAGGATTTC |
| | (SEQ ID NO: 33) |
| α Chain | NIQNPDPAVYQLRDSKSSDKSVCLFTDFDSQTNVSQSKDSD |
| Constant | VYITDKCVLDMRSMDFKSNSAVAWSNKSDFACANAFNNSI |
| region Amino | IPEDTFFPSPESSCDVKLVEKSFETDTNLNFQNLSVIGFRILL |
| Acid Sequence | LKVAGFNLLMTLRLWSS (SEQ ID NO: 18) |
| α Chain | ATATCCAGAACCCTGACCCTGCCGTGTACCAGCTGAGAG |
| Constant | ACTCTAAATCCAGTGACAAGTCTGTCTGCCTATTCACCG |
| region | ATTTTGATTCTCAAACAAATGTGTCACAAAGTAAGGATT |
| Nucleotide | CTGATGTGTATATCACAGACAAATGCGTGCTAGACATGA |
| Sequence | GGTCTATGGACTTCAAGAGCAACAGTGCTGTGGCCTGGA |
| | GCAACAAATCTGACTTTGCATGTGCAAACGCCTTCAACA |
| | ACAGCATTATTCCAGAAGACACCTTCTTCCCCAGCCCAG |
| | AAAGTTCCTGTGATGTCAAGCTGGTCGAGAAAAGCTTTG |
| | AAACAGATACGAACCTAAACTTTCAAAACCTGTCAGTGA |
| | TTGGGTTCCGAATCCTCCTCCTGAAAGTGGCCGGGTTTA |
| | ATCTGCTCATGACGCTGCGGCTGTGGTCCAGCTGA |
| | (SEQ ID NO: 19) |
| β Chain | EDLNKVFPPEVAVFEPSEAEISHTQKATLVCLATGFFPDHV |
| constant | ELSWWVNGKEVHSGVCTDPQPLKEQPALNDSRYCLSSRLR |
| region Amino | VSATFWQNPRNHFRCQVQFYGLSENDEWTQDRAKPVTQI |
| Acid | VSAEAWGRADCGFTSVSYQQGVLSATILYEILLGKATLYA |
| Sequence | VLVSALVLMAMVKRKDF (SEQ ID NO: 20) |
| β Chain | AGGACCTGAACAAGGTGTTCCCACCCGAGGTCGCTGTGT |
| constant | TTGAGCCATCAGAAGCAGAGATCTCCCACACCCAAAAG |
| region | GCCACACTGGTGTGCCTGGCCACAGGCTTCTTCCCTGAC |
| Nucleotide | CACGTGGAGCTGAGCTGGTGGGTGAATGGGAAGGAGGT |
| Sequence | GCACAGTGGGGTCTGCACGGACCCGCAGCCCCTCAAGG |
| | AGCAGCCCGCCCTCAATGACTCCAGATACTGCCTGAGCA |
| | GCCGCCTGAGGGTCTCGGCCACCTTCTGGCAGAACCCCC |
| | GCAACCACTTCCGCTGTCAAGTCCAGTTCTACGGGCTCT |
| | CGGAGAATGACGAGTGGACCCAGGATAGGGCCAAACCC |
| | GTCACCCAGATCGTCAGCGCCGAGGCCTGGGGTAGAGC |
| | AGACTGTGGCTTTACCTCGGTGTCCTACCAGCAAGGGGT |
| | CCTGTCTGCCACCATCCTCTATGAGATCCTGCTAGGGAA |
| | GGCCACCCTGTATGCTGTGCTGGTCAGCGCCCTTGTGTT |
| | GATGGCCATGGTCAAGAGAAAGGATTTC |
| | (SEQ ID NO: 21) |
| α Chain | QQKEVEQDPGPLSVPEGAIVSLNCTYSNSAFQYFMWYRQY |
| Variable | SRKGPELLMYTYSSGNKEDGRFTAQVDKSSKYISLFIRDSQ |
| region Amino | PSDSATYLCAMNRDDKIIFGKGTRLHILP |
| Acid Sequence | (SEQ ID NO: 14) |
| α Chain | CAGAAGGAGGTGGAGCAGGATCCTGGACCACTCAGTGT |
| Variable | TCCAGAGGGAGCCATTGTTTCTCTCAACTGCACTTACAG |
| region | CAACAGTGCTTTTCAATACTTCATGTGGTACAGACAGTA |
| Nucleic Acid | TTCCAGAAAAGGCCCTGAGTTGCTGATGTACACATACTC |
| Sequence | CAGTGGTAACAAAGAAGATGGAAGGTTTACAGCACAGG |
| | TCGATAAATCCAGCAAGTATATCTCCTTGTTCATCAGAG |
| | ACTCACAGCCCAGTGATTCAGCCACCTACCTCTGTGCAA |
| | TGAATCGGGATGACAAGATCATCTTTGGAAAAGGGACA |
| | CGACTTCATATTCTCCCCA (SEQ ID NO: 15) |
| β Chain | GVTQTPKFQVLKTGQSMTLQCAQDMNHNSMYWYRQDPG |
| Variable | MGLRLIYYSASEGTTDKGEVPNGYNVSRLNKREFSLRLESA |
| region Amino | APSQTSVYFCASSVSPGNYGYTFGSGTRLTVV |
| Acid Sequence | (SEQ ID NO: 16) |
| β Chain | AATGCTGGTGTCACTCAGACCCCAAAATTCCAGGTCCTG |
| Variable | AAGACAGGACAGAGCATGACACTGCAGTGTGCCCAGGA |
| Nucleic Acid | TATGAACCATAACTCCATGTACTGGTATCGACAAGACCC |
| Sequence | AGGCATGGGACTGAGGCTGATTTATTACTCAGCTTCTGA |
| | GGGTACCACTGACAAAGGAGAAGTCCCCAATGGCTACA |
| | ATGTCTCCAGATTAAACAAACGGGAGTTCTCGCTCAGGC |
| | TGGAGTCGGCTGCTCCCTCCCAGACATCTGTGTACTTCTG |
| | TGCCAGCAGTGTTTCCCCTGGAAACTATGGCTACACCTT |
| | CGGTTCGGGGACCAGGTTAACCGTTGTAG |
| | (SEQ ID NO: 17) |
| α Chain | QQKEVEQDPGPLSVPEGAIVSLNCTYSNSAFQYFMWYRQY |
| Variable and | SRKGPELLMYTYSSGNKEDGRFTAQVDKSSKYISLFIRDSQ |
| Constant | PSDSATYLCAMNRDDKIIFGKGTRLHILPNIQNPDPAVYQL |
| region Amino | RDSKSSDKSVCLFTDFDSQTNVSQSKDSDVYITDKCVLDM |
| Acid | RSMDFKSNSAVAWSNKSDFACANAFNNSIIPEDTFFPSPESS |
| Sequence | CDVKLVEKSFETDTNLNFQNLSVIGFRILLLKVAGFNLLMT |
| | LRLWSS (SEQ ID NO: 44) |
| α Chain | CAGAAGGAGGTGGAGCAGGATCCTGGACCACTCAGTGT |
| Variable and | TCCAGAGGGAGCCATTGTTTCTCTCAACTGCACTTACAG |
| Constant | CAACAGTGCTTTTCAATACTTCATGTGGTACAGACAGTA |
| region | TTCCAGAAAAGGCCCTGAGTTGCTGATGTACACATACTC |
| Nucleic Acid | CAGTGGTAACAAAGAAGATGGAAGGTTTACAGCACAGG |
| Sequence | TCGATAAATCCAGCAAGTATATCTCCTTGTTCATCAGAG |
| | ACTCACAGCCCAGTGATTCAGCCACCTACCTCTGTGCAA |
| | TGAATCGGGATGACAAGATCATCTTTGGAAAAGGGACA |
| | CGACTTCATATTCTCCCCAATATCCAGAACCCTGACCCT |
| | GCCGTGTACCAGCTGAGAGACTCTAAATCCAGTGACAAG |
| | TCTGTCTGCCTATTCACCGATTTTGATTCTCAAACAAATG |
| | TGTCACAAAGTAAGGATTCTGATGTGTATATCACAGACA |
| | AATGCGTGCTAGACATGAGGTCTATGGACTTCAAGAGCA |
| | ACAGTGCTGTGGCCTGGAGCAACAAATCTGACTTTGCAT |
| | GTGCAAACGCCTTCAACAACAGCATTATTCCAGAAGACA |
| | CCTTCTTCCCCAGCCCAGAAAGTTCCTGTGATGTCAAGC |
| | TGGTCGAGAAAAGCTTTGAAACAGATACGAACCTAAAC |
| | TTTCAAAACCTGTCAGTGATTGGGTTCCGAATCCTCCTCC |
| | TGAAAGTGGCCGGGTTTAATCTGCTCATGACGCTGCGGC |
| | TGTGGTCCAGCTGA (SEQ ID NO: 45) |
| β Chain | GVTQTPKFQVLKTGQSMTLQCAQDMNHNSMYWYRQDPG |
| Variable and | MGLRLIYYSASEGTTDKGEVPNGYNVSRLNKREFSLRLESA |
| Constant | APSQTSVYFCASSVSPGNYGYTFGSGTRLTVVEDLNKVFPP |
| region Amino | EVAVFEPSEAEISHTQKATLVCLATGFFPDHVELSWWVNG |
| Acid | KEVHSGVCTDPQPLKEQPALNDSRYCLSSRLRVSATFWQN |
| Sequence | PRNHFRCQVQFYGLSENDEWTQDRAKPVTQIVSAEAWGR |
| | ADCGFTSVSYQQGVLSATILYEILLGKATLYAVLVSALVLM |
| | AMVKRKDF (SEQ ID NO: 46) |
| β Chain | AATGCTGGTGTCACTCAGACCCCAAAATTCCAGGTCCTG |
| Variable and | AAGACAGGACAGAGCATGACACTGCAGTGTGCCCAGGA |
| Constant | TATGAACCATAACTCCATGTACTGGTATCGACAAGACCC |
| region | AGGCATGGGACTGAGGCTGATTTATTACTCAGCTTCTGA |
| Nucleic Acid | GGGTACCACTGACAAAGGAGAAGTCCCCAATGGCTACA |
| Sequence | ATGTCTCCAGATTAAACAAACGGGAGTTCTCGCTCAGGC |
| | TGGAGTCGGCTGCTCCCTCCCAGACATCTGTGTACTTCTG |
| | TGCCAGCAGTGTTTCCCCTGGAAACTATGGCTACACCTT |
| | CGGTTCGGGGACCAGGTTAACCGTTGTAGAGGACCTGAA |
| | CAAGGTGTTCCCACCCGAGGTCGCTGTGTTTGAGCCATC |
| | AGAAGCAGAGATCTCCCACACCCAAAAGGCCACACTGG |
| | TGTGCCTGGCCACAGGCTTCTTCCCTGACCACGTGGAGC |
| | TGAGCTGGTGGGTGAATGGGAAGGAGGTGCACAGTGGG |
| | GTCTGCACGGACCCGCAGCCCCTCAAGGAGCAGCCCGCC |
| | CTCAATGACTCCAGATACTGCCTGAGCAGCCGCCTGAGG |
| | GTCTCGGCCACCTTCTGGCAGAACCCCCGCAACCACTTC |
| | CGCTGTCAAGTCCAGTTCTACGGGCTCTCGGAGAATGAC |
| | GAGTGGACCCAGGATAGGGCCAAACCCGTCACCCAGAT |
| | CGTCAGCGCCGAGGCCTGGGGTAGAGCAGACTGTGGCTT |
| | TACCTCGGTGTCCTACCAGCAAGGGGTCCTGTCTGCCAC |
| | CATCCTCTATGAGATCCTGCTAGGGAAGGCCACCCTGTA |
| | TGCTGTGCTGGTCAGCGCCCTTGTGTTGATGGCCATGGT |
| | CAAGAGAAAGGATTTC (SEQ ID NO: 47) |
| Clonotype ID | CSVGQGLANVLTF (SEQ ID NO: 34) |
| 1 TCR B | |
| CDR3 | |
| Clonotype ID | CAVRNTDKLIF (SEQ ID NO: 35) |
| 1 TCR A | |
| CDR3 | |
| Clonotype ID | CASSQWLGAETQYF (SEQ ID NO: 36) |
| 3 TCR B | |
| CDR3 | |
| Clonotype ID | CAFRDNYGQNFVF (SEQ ID NO: 37) |
| 3 TCR A | |
| CDR3 | |
| Clonotype ID | CASSPGDEQYF (SEQ ID NO: 38) |
| 4 TCR B | |
| CDR3 | |
| Clonotype ID | CAGKTSYDKVIF (SEQ ID NO: 39) |
| 4 TCR A | |
| CDR3 | |
| Clonotype ID | CSAPRRERLDEQFF (SEQ ID NO: 40) |
| 5 TCR B | |
| CDR3 | |
| Clonotype ID | CAEVNNNARLMF (SEQ ID NO: 41) |
| 5 TCR A | |
| CDR3 | |
| Clonotype ID | CAMREGGDTGFQKLVF (SEQ ID NO: 42) |
| 5 TCR A | |
| CDR3 | |
| Clonotype ID | CASSVSPGNYGYTF (SEQ ID NO: 43) |
| 2 TCR B | |
| CDR3 | |

For example, the international ImMunoGeneTics information System® (www.imgt.org) is based on the IMGT numbering for all immunoglobulin and T cell receptor V-REGIONs of all species (IMGT®, the international ImMunoGeneTics information System®; Lefranc M I P et al., (1991) Nucleic Acids Res. 27(1): 209-12; Ruiz M et al., (2000) Nucleic Acids Res. 28(1): 219-21; Lefranc M P (2001) Nucleic Acids Res. 29(1): 207-9; Lefranc M P (2003) Nucleic Acids Res. 31(1): 307-10; Lefranc M P et al., (2005) Dev. Comp. Immunol. 29(3): 185-203; Kaas Q et al., (2007) Briefings in Functional Genomics & Proteomics, 6(4): 253-64).

The abYsis algorithm compiles antibody protein sequences from EMBLIG, which contains immunoglobulin information extracted from the EMBL-ENA databank, the Kabat collection, and the Protein Databank (Swindells M B et al., (2017) J Mol Biol. 2017 Feb. 3; 429(3):356-364).

In some aspects, provided herein is an engineered TCRs capable of binding to epidermal growth factor receptor T790M mutation on human leukocyte antigen-A (HLA-A)*02:01 (e.g., comprising the amino acid sequence of SEQ ID NO: 1). In some aspects, the engineered TCR comprises a TCR beta chain comprising (i) a CDR1β comprising the amino acid sequence set forth in SEQ ID NO: 5, (ii) a CDR2β comprising the amino acid sequence set forth in SEQ ID NO:6, and (iii) a CDR3β comprising the amino acid sequence set forth in SEQ ID NO: 7, and a TCR alpha chain comprising (i) CDR1α comprising the amino acid sequence set forth in SEQ ID NO: 2, (ii) a CDR2α comprising the amino acid sequence set forth in SEQ ID NO: 3, and (iii) a CDR3α comprising the amino acid sequence set forth in SEQ ID NO: 4. In some aspects, the engineered TCR comprises an antigen ligand-binding domain capable of binding to CD3.

In some aspects, provided herein is an engineered TCRs capable of to an epidermal growth factor receptor (EGFR) comprising a T790M mutation on human leukocyte antigen-A (HLA-A)*02:01 (e.g., comprising SEQ ID NO: 1), wherein the engineered TCRs comprises: i) a CDR1β, a CDR2β, and a CDR3β of SEQ ID NO: 16; and ii) a CDR1α, a CDR2α, and a CDR3α of SEQ ID NO: 14.

In some aspects, the engineered TCR comprises a CDR3β of SEQ ID NO: 34, 36, 38, 40, or 43. In some aspects, the engineered TCR comprises a CDR3α of SEQ ID NO: 35, 37, 39, 41, or 42.

In some aspects, the CDRs are the Kabat-defined CDRs, the Chothia-defined CDRs, the AbM-defined CDRs, or the IMGT-defined CDRs.

In some aspects, the CDRs of engineered TCRs can be determined according to the Chothia numbering scheme, which refers to the location of immunoglobulin structural loops (see, e.g., Chothia C &Lesk A M, (1987), J Mol Biol 196: 901-917; Al-Lazikani B et al., (1997) J Mol Biol 273: 927-948; Chothia C et al., (1992) J Mol Biol 227: 799-817; Tramontano A et al., (1990) J Mol Biol 215(1): 175-82; and U.S. Pat. No. 7,709,226). Typically, when using the Kabat numbering convention, the Chothia CDR-H1 loop is present at heavy chain amino acids 26 to 32, 33, or 34, the Chothia CDR-H2 loop is present at heavy chain amino acids 52 to 56, and the Chothia CDR-H3 loop is present at heavy chain amino acids 95 to 102, while the Chothia CDR-L1 loop is present at light chain amino acids 24 to 34, the Chothia CDR-L2 loop is present at light chain amino acids 50 to 56, and the Chothia CDR-L3 loop is present at light chain amino acids 89 to 97. The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34).

In some aspects, the CDRs of engineered TCRs can be determined according to the IMGT numbering system as described in Lefranc M-P, (1999) The Immunologist 7: 132-136 and Lefranc M-P et al., (1999) Nucleic Acids Res 27: 209-212. According to the IMGT numbering scheme, CDR1B is at positions 26 to 35, CDR2β is at positions 51 to 57, CDR3β is at positions 93 to 102, CDR1A is at positions 27 to 32, CDR2A is at positions 50 to 52, and CDR3A is at positions 89 to 97. In some aspects, provided herein are antibodies and antigen-binding fragments thereof that specifically bind to EGFR comprising a T790M mutation on HLA-A*2:01 and comprise the IMGT ap and a R CDRs of a TCR in table 1, for example, as described in Lefranc M-P (1999) supra and Lefranc M-P et al., (1999) supra).

In some aspects, the CDRs of engineered TCRs can be determined according to MacCallum R M et al., (1996) J Mol Biol 262: 732-745. See also, e.g., Martin A. “Protein Sequence and Structure Analysis of Antibody Variable Domains,” in Antibody Engineering, Kontermann and Dubel, eds., Chapter 31, pp. 422-439, Springer-Verlag, Berlin (2001). In some aspects, provided herein are antibodies or antigen-binding fragments thereof that specifically bind to EGFR comprising a T790M mutation on HLA-A*2:01 are determined by the method in MacCallum R M et al.

In some aspects, the CDRs of engineered TCRs can be determined according to the AbM numbering scheme, which refers AbM hypervariable regions which represent a compromise between the Kabat CDRs and Chothia structural loops, and are used by Oxford Molecular's AbM antibody modeling software (Oxford Molecular Group, Inc.). In some aspects, provided herein are antibodies or antigen-binding fragments thereof that specifically bind to epidermal growth factor receptor (EGFR) comprising a T790M mutation on human leukocyte antigen-A (HLA-A)*02:01 are determined by the AbM numbering scheme.

In some aspects, the engineered TCR comprise a beta chain (TCR-β) and an alpha chain (TCR-α), wherein the TCR-β comprises an amino acid sequence having at least 85%, at least 90%, at least 91% at least 92%, at least 93%, at least 94%, at least 95%, at least 96%, at least 97%, at least 98%, at least 99%, or at least 100% sequence identity to SEQ ID NO: 32 and the TCR-α comprises an amino acid sequence having at least 85%, at least 90%, at least 91%, at least 92%, at least 93%, at least 94%, at least 95%, at least 96%, at least 97%, at least 98%, at least 99%, or at least 100% sequence identity to SEQ ID NO: 30.

In some aspects, the engineered TCR comprises a beta chain (TCR-β) and an alpha chain (TCR-α), wherein the TCR-β comprises an amino acid sequence having at least 85%, at least 90%, at least 91% at least 92%, at least 93%, at least 94%, at least 95%, at least 96%, at least 97%, at least 98%, at least 99%, or at least 100% sequence identity to SEQ ID NO: 46 and the TCR-α comprises an amino acid sequence having at least 85%, at least 90%, at least 91%, at least 92%, at least 93%, at least 94%, at least 95%, at least 96%, at least 97%, at least 98%, at least 99%, or at least 100% sequence identity to SEQ ID NO: 44.

In some aspects, the engineered TCR comprises a TCR-β chain comprising an amino acid sequence at least 85% identical to the amino acid sequence SEQ ID NO: 32. In some aspects, the engineered TCR comprises a TCR-β chain comprising an amino acid sequence at least 90% identical to the amino acid sequence SEQ ID NO: 32. In some aspects, the engineered TCR comprises a TCR-β chain comprising an amino acid sequence at least 95% identical to the amino acid sequence SEQ ID NO: 32. In some aspects, the engineered TCR comprises a TCR-β chain comprising an amino acid sequence at least 98% identical to the amino acid sequence SEQ ID NO: 32. In some aspects, the engineered TCR comprises a TCR-β chain comprising an amino acid sequence at least 99% identical to the amino acid sequence SEQ ID NO: 32. In some aspects, the engineered TCR comprises a TCR-β chain comprising an amino acid sequence identical to the amino acid sequence SEQ ID NO: 32. In some aspects, the engineered TCR comprises a TCR-α chain comprising an amino acid sequence at least 85% identical to the amino acid sequence of SEQ ID NO: 30. In some aspects, the engineered TCR comprises a TCR-α comprising an amino acid sequence at least 90% identical to the amino acid sequence of SEQ ID NO: 30. In some aspects, the engineered TCR comprises a TCR-α comprising an amino acid sequence at least 95% identical to the amino acid sequence of SEQ ID NO: 30. In some aspects, the engineered TCR comprises a TCR-α comprising an amino acid sequence at least 98% identical to the amino acid sequence of SEQ ID NO: 30. In some aspects, the engineered TCR comprises a TCR-α comprising an amino acid sequence at least 99% identical to the amino acid sequence of SEQ ID NO: 30. In some aspects, the engineered TCR comprises a TCR-α comprising an amino acid sequence identical to the amino acid sequence of SEQ ID NO: 30.

In some aspects, the engineered TCR comprises an amino acid sequence having at least 85%, at least 90%, at least 91%, at least 92%, at least 93%, at least 94%, at least 95%, at least 96%, at least 97%, at least 98%, at least 99%, or at least 100% sequence identity to SEQ ID NO: 28.

In some aspects, the engineered TCRs is humanized or chimeric.

In some aspects, the engineered TCR of the disclosure comprises an extracellular antigen-binding domain.

In some aspects, the disclosure also provides nucleic acid molecules encoding one or more of the engineered TCR constructs disclosed herein. In some aspects, the disclosure includes a vector into which the DNA encoding one or more of the engineered TCR constructs disclosed herein is inserted. In some aspects, the nucleic acids can be cloned into vectors including but not limited to plasmids, phagemids, phage derivatives, animal viruses, and cosmids. Viruses that can be used as vectors include, but are not limited to, retrovirus, adenovirus, adeno-associated virus, herpes virus, and lentivirus. The present invention is not limited to the use of constitutive promoters, while inducible promoters are also considered. The use of an inducible promoter provides a molecular switch that can initiate the expression of an operably linked polynucleotide sequence when expression is required, or close the expression when expression is not required. Examples of inducible promoters include, but are not limited to, NFAT6 promoter, metallothionein promoter, glucocorticoid promoter, progesterone promoter, and tetracycline-regulated promoter.

Certain aspects of the disclosure are directed to a polynucleotide comprising a nucleic acid encoding a TCR-β chain disclosed herein.

Certain aspects of the disclosure are directed to polynucleotide comprising a nucleic acid encoding a TCR-α chain disclosed herein.

Certain aspects of the disclosure are directed to a polynucleotide comprising a nucleic acid encoding a TCR-β chain and a TCR-α chain disclosed herein.

In some aspects, the engineered TCR-β comprises an antigen-binding domain variable region which is encoded by a polynucleotide sequence comprising a nucleic acid sequence having at least 85%, at least 90%, at least 91%, at least 92%, at least 93%, at least 94%, at least 95%, at least 96%, at least 97%, at least 98%, at least 99%, or at least 100% sequence identity to SEQ ID NO: 17.

In some aspects, the engineered TCR-α comprises an antigen-binding domain variable region which is encoded by a polynucleotide sequence comprising a nucleic acid sequence having at least 85%, at least 90%, at least 91%, at least 92%, at least 93%, at least 94%, at least 95%, at least 96%, at least 97%, at least 98%, at least 99%, or at least 100% sequence identity to SEQ ID NO: 15.

In some aspects, the engineered TCR-β comprises an antigen-binding domain variable region and a constant region which are encoded by a polynucleotide sequence comprising a nucleic acid sequence having at least 85%, at least 90%, at least 91%, at least 92%, at least 93%, at least 94%, at least 95%, at least 96%, at least 97%, at least 98%, at least 99%, or at least 100% sequence identity to SEQ ID NO: 47.

In some aspects, the engineered TCR-α comprises a antigen-binding domain variable region and a constant region which are encoded by a polynucleotide sequence comprising a nucleic acid sequence having at least 85%, at least 90%, at least 91%, at least 92%, at least 93%, at least 94%, at least 95%, at least 96%, at least 97%, at least 98%, at least 99%, or at least 100% sequence identity to SEQ ID NO: 45.

In some aspects, the engineered TCR-β comprises a signal sequence, antigen-binding domain variable region and constant region which are encoded by a polynucleotide sequence comprising a nucleic acid sequence having at least 85%, at least 90%, at least 91%, at least 92%, at least 93%, at least 94%, at least 95%, at least 96%, at least 97%, at least 98%, at least 99%, or at least 100% sequence identity to SEQ ID NO: 33.

In some aspects, the engineered TCR-α comprises a signal sequence, antigen-binding domain variable region and constant region which are encoded by a polynucleotide sequence comprising a nucleic acid sequence having at least 85%, at least 90%, at least 91%, at least 92%, at least 93%, at least 94%, at least 95%, at least 96%, at least 97%, at least 98%, at least 99%, or at least 100% sequence identity to SEQ ID NO: 31.

Certain aspects of the disclosure are directed to a polynucleotide comprising a nucleic acid sequence encoding an engineered TCR comprising an amino acid sequence having at least 85%, at least 90%, at least 91%, at least 92%, at least 93%, at least 94%, at least 95%, at least 96%, at least 97%, at least 98%, at least 99%, or at least 100% sequence identity to SEQ ID NO: 29.

Certain aspects of the disclosure are directed to a vector comprising a polynucleotide disclosed herein.

Biological methods for introducing polynucleotides of interest into host cells include the use of DNA and RNA vectors. Viral vectors, especially retroviral vectors, have become the most widely used method for inserting genes into mammalian, such as human cells. Other viral vectors can be derived from lentivirus, poxvirus, herpes simplex virus I, adenovirus and adeno-associated virus.

Certain aspects of the disclosure are directed to a host cell comprising a polynucleotide disclosed herein.

In some aspects, the host cell is selected from the group consisting of CHO, HEK-293T, HeLa and BHK cells, optionally wherein the CHO cell is a CHO-K1SP cell.

### IV. TCR T-Cells

The present disclosure provides immune cells (e.g., T cells) modified to comprise a T cell receptor sequence disclosed herein. In some aspects the TCR T cell is capable of recognizing EGFR(T790M) mutations when presented on the HLA-A*02:01 allele (MQLMPFGCLL (SEQ ID NO:1)) of a cancer cell. In some aspects, the T cell is capable of killing the cancer cell.

In some aspects, before expansion and genetic modification, a source of T cells is obtained from a subject. Examples of subjects include mammals such as humans, primates, dogs, cats, mice, rats, and transgenic species thereof. T cells can be obtained from many sources, including peripheral blood mononuclear cells, bone marrow, lymph node tissue, umbilical cord blood, thymus tissue, tissue from the site of infection, ascites, pleural effusion, spleen tissue, and tumors. In certain aspects of the disclosure, any number of T cell lines available in the art can be used. In some aspects, any number of techniques known to a skilled person, such as Ficoll separation technology, can be used to obtain T cells from blood units collected from a subject. In some aspects, cells from the circulating blood of an individual are obtained by apheresis. Products obtained by apheresis blood apheresis usually contain lymphocytes, including T cells, monocytes, granulocytes, B cells, other nucleated white blood cells, red blood cells and platelets.

In some aspects, the cells collected by apheresis can be washed to remove the plasma fraction and placed in an appropriate buffer or medium for subsequent processing steps. Multiple rounds of selection can also be used in the context of the invention. In some aspects, it may be necessary to perform a selection procedure and use “unselected” cells during activation and expansion. “Unselected” cells can also undergo other rounds of selection.

In some aspects, the T cell of the present disclosure can be expanded by contacting with a surface to which an agent that stimulates the CD3/TCR complex-related signal and a ligand that stimulates a costimulatory molecule on the surface of the T cell are attached. In particular, the T cell population can be stimulated, for example by contacting with an anti-CD3 antibody or antigen-binding fragments thereof or an anti-CD2 antibody immobilized on a surface, or by contacting a protein kinase C activator (for example, bryostatin) and calcium ionophore.

CD3 (Cluster of Differentiation 3) T cell co-receptor is a protein complex composed of four different chains. In mammals, the complex contains one CD3.gamma. chain, CD3.delta. chain, and two CD3 epsilon. chains. These chains have a molecule of accessory T cell receptor (TCR) and zeta-chain to generate activation signals for T lymphocytes. The TCR, zeta. chain and CD3 molecule together constitute a T cell receptor complex. The CD3 molecule is connected to the T cell receptor (TCR) through a salt bridge to form a TCR-CD3 complex, which participates in the signaling of T cells, and is mainly used to label thymocytes, T lymphocytes and T cell lymphomas. The cytoplasmic segment of CD3 contains immunoreceptor tyrosine-based activation motif (ITAM). TCR recognizes and binds to the antigen peptide presented by the MHC (major histo-compatibility complex) molecule, resulting in the tyrosine residues in the conserved sequence of ITAM of CD3. Being phosphorylated by the tyrosine protein kinase p56lck in T cells, and then recruiting other tyrosine protein kinases (such as ZAP-70) containing SH2 (Scr homology 2) domain. The phosphorylation of ITAM and the binding to ZAP-70 are one of the important biochemical reactions in the early stages of the signaling process of T cell activation. Therefore, the function of the CD3 molecule is to transduce the activation signal generated by the TCR to recognize the antigen. In this application, the exogenous receptor that can bind to the target antigen and trigger CD3 signal activation includes at least one CD3 binding site and at least one additional antigen binding site specific to bacterial substance, viral protein, autoimmune marker, or antigen present specific cells (e.g., cell surface proteins of B cells, T cells, natural killer (NK) cells, bone marrow cells, phagocytes, or tumor cells). Such exogenous receptors can cross-link two kinds of cells and can be used to direct T cells to specific targets and trigger the cytotoxic activity of T cells on the target cells. Examples of such targets may be tumor cells or infectious agents, such as viral pathogens or bacterial pathogens.

Prior to in vitro manipulation or genetic modification of the immune effector cells disclosed herein, the source of cells can be obtained from a subject. In particular, the immune effector cells for use with the TCRs as disclosed herein comprise T cells. In some aspects, the T cells are obtained from peripheral blood mononuclear cells, bone marrow, lymph nodes tissue, cord blood, thymus issue, tissue from a site of an infection, ascites, pleural effusion, spleen tissue, or a tumor. In some aspects, the T cells are obtained from peripheral blood mononuclear cells. In some aspects, the T cells are obtained from tumor cells (e.g., NSCLC cells). In some aspects, T cells can be obtained from a unit of blood collected from the subject using any number of techniques known to the skilled person, such as FICOLL separation. In some aspects, cells from the circulating blood of an individual are obtained by apheresis. The apheresis product typically contains lymphocytes, including T cells, monocytes, granulocyte, B cells, other nucleated white blood cells, red blood cells, and platelets. In some aspects, the cells collected by apheresis can be washed to remove the plasma fraction and to place the cells in an appropriate buffer or media for subsequent processing. In some aspects, the cells are washed with PBS. In some aspects, the washed solution lacks calcium, and can lack magnesium or can lack many, if not all, divalent cations. As would be appreciated by those of ordinary skill in the art, a washing step can be accomplished by methods known to those in the art, such as by using a semiautomated flowthrough centrifuge. After washing, the cells can be resuspended in a variety of biocompatible buffers or other saline solution with or without buffer. In some aspects, the undesirable components of the apheresis sample can be removed in the cell directly resuspended culture media.

In some aspects, T cells are isolated from peripheral blood mononuclear cells (PBMCs) by lysing the red blood cells and depleting the monocytes, for example, by centrifugation through a PERCOLL™ gradient. A specific subpopulation of T cells, such as CD28+, CD4+, CD8+, CD45RA+, and CD45RO+ T cells, can be further isolated by positive or negative selection techniques. For example, enrichment of a T cell population by negative selection can be accomplished with a combination of antibodies directed to surface markers unique to the negatively selected cells. One method for use herein is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD1b, CD16, HLA-DR, and CD8. Flow cytometry and cell sorting can also be used to isolate cell populations of interest for use in the present disclosure.

PBMCs can be used directly for genetic modification with the various methods as disclosed herein. In some aspects, after isolation of PBMC, T lymphocytes are further isolated and in some aspects, both cytotoxic and helper T lymphocytes can be sorted into naive, memory, and effector T cell subpopulations either before or after genetic modification and/or expansion. CD8+ cells can be obtained by using standard methods. In some aspects, CD8+ cells are further sorted into naive, central memory, and effector cells by identifying cell surface antigens that are associated with each of those types of CD8+ cells. In some aspects, memory T cells are present in both CD62L+ and CD62L-subsets of CD8+ peripheral blood lymphocytes. PBMC are sorted into CD62L-CD8+ and CD62L+CD8+ fractions after staining with anti-CD8 and anti-CD62L antibodies. In some aspects, the expression of phenotypic markers of central memory TCM include CD45RO, CD62L, CCR7, CD28, CD3, and CD127 and are negative for granzyme B. In some aspects, central memory T cells are CD45RO+, CD62L+, CD8+ T cells. In some aspects, effector T cells are negative for CD62L, CCR7, CD28, and CD127, and positive for granzyme B and perforin. In some aspects, naive CD8+ T lymphocytes are characterized by the expression of phenotypic markers of naive T cells including CD62L, CCR7, CD28, CD3, CD 127, and CD45RA.

In some aspects, CD4+ T cells are further sorted into subpopulations. For example, CD4+ T helper cells can be sorted into naive, central memory, and effector cells by identifying cell populations that have cell surface antigens. CD4+ lymphocytes can be obtained by standard methods.

The immune effector cells, such as T cells, can be genetically modified following isolation using known methods, or the immune effector cells can be activated and expanded (or differentiated in the case of progenitors) in vitro prior to being genetically modified. In another embodiment, the immune effector cells, such as T cells, are genetically modified with the T cell receptors disclosed herein (e.g., transduced with a viral vector comprising a nucleic acid encoding an a and 8 chain) and then are activated and expanded in vitro. Methods for activating and expanding T cells are known in the art and are disclosed, for example, in U.S. Pat. Nos. 6,905,874; 6,867,041; 6,797,514; WO2012079000. Generally, such methods include contacting PBMC or isolated T cells with a stimulatory agent and costimulatory agent, such as anti-CD3 and anti-CD28 antibodies, generally attached to a bead or other surface, in a culture medium with appropriate cytokines, such as IL-2. Anti-CD3 and anti-CD28 antibodies attached to the same bead serve as a “surrogate” antigen presenting cell (APC). In some aspects, the T cells can be activated and stimulated to proliferate with feeder cells and appropriate antibodies and cytokines using methods such as those disclosed in U.S. Pat. Nos. 6,040,177; 5,827,642; and WO2012129514.

In some aspects, the cells are formulated by first harvesting them from their culture medium, and then washing and concentrating the cells in a medium and container system suitable for administration (a “pharmaceutically acceptable” carrier) in a treatment-effective amount. Suitable infusion medium can be any isotonic medium formulation, typically normal saline, Normosol R (Abbott) or Plasma-Lyte A (Baxter), but also 5% dextrose in water or Ringer's lactate can be utilized. The infusion medium can be supplemented with human serum albumin.

The cells can be autologous or heterologous to the patient undergoing therapy. If desired, the treatment can also include administration of mitogens (e.g., PHA) or lymphokines, cytokines, and/or chemokines (e.g., IFN-7, IL-2, IL-12, TNF-α, IL-18, and TNF-β, GM-CSF, IL-4, IL-13, Flt3-L, RANTES, MIP1α, etc.) as disclosed herein to enhance induction of the immune response.

Humoral immune responses, mediated primarily by helper T cells capable of activating B cells thus leading to antibody production, may be induced. A variety of techniques may be used for analyzing the type of immune responses induced by the compositions of the present disclosure, which are well disclosed in the art; e.g., Current Protocols in Immunology, Edited by: John E. Coligan, Ada M. Kruisbeek, David H. Margulies, Ethan M. Shevach, Warren Strober (2001) John Wiley & Sons, N.Y., N.Y.

In some aspects, the immune cell is a T cell. In some aspects, the immune cell is a T lymphocyte selected from an inflammatory T lymphocyte, a cytotoxic T lymphocyte, a regulatory T lymphocyte, or a helper T lymphocyte. In some cases, the immune cell is a CD8+ cytotoxic T lymphocyte.

Whether prior to or after genetic modification of the engineered cells (e.g., T cells), even if the genetically modified immune cells of the present disclosure are activated and proliferate independently of antigen binding mechanisms, the immune cells, particularly T-cells of the present disclosure can be further activated and expanded generally using methods as disclosed, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005. T cells can be expanded in vitro or in vivo.

In some aspects, the T cells of the disclosure are expanded by contact with an agent that stimulates a CD3 TCR complex and a costimulatory molecule on the surface of the T cells to create an activation signal for the T-cell. For example, chemicals such as calcium ionophore A23187, phorbol 12-myristate 13-acetate (PMA), or mitogenic lectins like phytohemagglutinin (PHA) can be used to create an activation signal for the T-cell.

As non-limiting examples, T cell populations can be stimulated in vitro such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For costimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. Conditions appropriate for T cell culture include an appropriate media (e.g., Minimal Essential Media or RPMI Media 1640 or, X-vivo 5, (Lonza)) that can contain factors necessary for proliferation and viability, including serum (e.g., fetal bovine or human serum), interleukin-2 (IL-2), insulin, IFN-g, IL-4, IL-7, GM-CSF, IL-10, IL-2, IL-15, TGFp, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, A1M-V, DMEM, MEM, a-MEM, F-12, X-Vivo 1, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% 02). T cells that have been exposed to varied stimulation times can exhibit different characteristics.

In some aspects, the cells can be expanded by co-culturing with tissue or cells. The cells can also be expanded in vivo, for example in the subject's blood after administrating said cell into the subject.

In some aspects, provided herein is a method of treating cancer or a tumor in an subject, the method comprising administering to the individual a therapeutically effective amount of any of the of the engineered TCR-T cells disclosed herein.

In some aspects, the subject is a human.

In some aspects, the cancer is lung cancer.

In some aspects, the cancer is non-small cell lung cancer (NSCLC).

In some aspects, the cancer or tumor comprises an epidermal growth factor receptor (EGFR) T790M mutation on human leukocyte antigen-A (HLA-A)*02:01.

In some aspects, the subject suffers from a cancer or tumor (e.g., NSCLC) which is epidermal growth factor receptor (EGFR) tyrosine kinase inhibitor (EGFR-TKI) resistant.

### V. Bispecific T Cell Engagers (BiTEs)

Certain aspects of the disclosure are directed to a bispecific T cell engager (BiTE) comprising an engineered T-cell receptor (TCR) or antigen binding fragment thereof disclosed herein. In some aspects, the BiTE is capable of binding to an epidermal growth factor receptor (EGFR) comprising a T790M, mutation on human leukocyte antigen-A (HLA-A)*02:01 (e.g., SEQ ID NO: 1).

A bi-specific antibody molecule as employed herein refers to a molecule with two antigen binding domains, which may bind the same or different antigens. A BiTE is a subclass of bispecific antibody molecules. “Non-IgG-like” antibodies include antibodies that lack an Fc portion such as bispecific T cell engagers (BiTE), DART, tetravalent antiparallel structures (TandAbs) and VH-only bi-nanobodies. The non-IgG-like antibodies due to their lack of an Fc portion are smaller and have shorter in vivo half-lives.

As used herein, the term “Bi-specific T-cell engagers”, or “BiTEs”, refer to a class of artificial bispecific monoclonal antibodies that are investigated for the use as anti-cancer drugs. They direct a host's immune system, more specifically the T cells' cytotoxic activity, against cancer cells. BiTEs are fusion proteins consisting of two single-chain variable fragments (scFvs) of different antibodies, or amino acid sequences from four different genes, on a single peptide chain of about 55 kilodaltons. One of the scFvs binds to T cells via the CD3 receptor, and the other to a tumor cell via a tumor specific molecule. Like other bispecific antibodies, and unlike ordinary monoclonal antibodies, BiTEs form a link between T cells and tumor cells. This causes T cells to exert cytotoxic activity on tumor cells by producing proteins like perforin and granzymes, independently of the presence of MHC I or co-stimulatory molecules. These proteins enter tumor cells and initiate the cell's apoptosis. This action mimics physiological processes observed during T cell attacks against tumor cells. BiTE is a registered trademark of Micromet AG (fully owned subsidiary of Amgen Inc).

BiTEs are able to form a link between T cells and tumour cells by virtue of their specificities for an antigen on the T cell and an antigen on the tumour cell. This leads to activation of the T-cells and triggers the T cells to exert their cytotoxic effects on tumour cells, independently of MHC I or co-stimulatory molecules.

In some aspects, the BiTe harbors specificity towards antibody or antigen fragments targets expressed on the surface of cells (e.g., cancer cells or targeted epitopes of interest) and exhibit specificity for CD3 coreceptor of a T cell in addition to the disclosed engineered or antigen binding fragment disclosed herein.

In some aspects, the present disclosure BiTE is capable of activation of antigen specific T cells, which can kill targeted cancer cells expressing a particular epitopes of interest (e.g., EGFR comprising a T790M, mutation on human leukocyte antigen-A (HLA-A)*02:01 (e.g., SEQ ID NO: 1)).

In some aspects, the BiTE is specific for at least a surface antigen on a T cell of interest. Examples of T cell surface antigens include but are not limited to: CD3, CD2, VLA-1, CD8, CD4, CCR6, CXCR5, CD25, CD31, CD45RO, CD197, CD127, CD38, CD27, CD196, CD277 and CXCR3, particularly CD2, CD3, CD31 and CD277.

In some aspects, the BiTE comprises (i) a binding region specific to a surface antigen on a T cell of interest (e.g., selected from CD3 (such as CD3 delta, CD3 epsilon or CD3 gamma), and (ii) an antigen binding region (e.g., specific to an antigen comprising a T790M, mutation on human leukocyte antigen-A (HLA-A)*02:01 (e.g., SEQ ID NO: 1)) comprising TCR-α chain and TCR-β chain disclosed herein.

In some aspects, the immune cell engager (such as T cell engager) is arranged is the format VL1-linker1-VH1-linker1-VH2-linker3-VL2.

In some aspects, the BiTEs disclosed herein direct T-cells to NSCLC cells.

In some aspects, provided herein is a method of treating cancer or a tumor in an subject, the method comprising administering to the individual a therapeutically effective amount of any of the of the BiTE disclosed herein.

In some aspects, the subject is a human.

In some aspects, the cancer is lung cancer.

In some aspects, the cancer is non-small cell lung cancer (NSCLC).

In some aspects, the subject suffers from a cancer or tumor (e.g., NSCLC) which is epidermal growth factor receptor (EGFR) tyrosine kinase inhibitor (EGFR-TKI) resistant.

### VI. Antibodies

In some aspects, provided herein are antibodies (e.g. monoclonal antibodies) and antigen-binding fragments thereof which specifically bind to a Epidermal growth factor receptor (EGFR) epitope comprising a T790M mutation on human leukocyte antigen-A (HLA-A)*02:01 MQLMPFGCLL (SEQ ID NO: 1).

In some aspects, an antibody or antigen-binding fragment thereof provided herein comprises six CDRs (e.g., a CDR H1, a CDR H2, a CDR H3, a CDR L1, a CDR L2, and a CDR L3). The CDRs can be determined by a number of algorithms in the art, such as IMGT, abYsis, and Kabat.

For example, the international ImMunoGeneTics information System® (http://www.imgt.org) is based on the IMGT numbering for all immunoglobulin and T cell receptor V-REGIONs of all species (IMGT®, the international ImMunoGeneTics information System®; Lefranc M P et al., (1991) Nucleic Acids Res. 27(1): 209-12; Ruiz M et al., (2000) Nucleic Acids Res. 28(1): 219-21; Lefranc M P (2001) Nucleic Acids Res. 29(1): 207-9; Lefranc M P (2003) Nucleic Acids Res. 31(1): 307-10; Lefranc M P et al., (2005) Dev. Comp. Immunol. 29(3): 185-203; Kaas Q et al., (2007) Briefings in Functional Genomics & Proteomics, 6(4): 253-64).

The abYsis algorithm compiles antibody protein sequences from EMBLIG which contains antibody information extracted from the EMBL-ENA databank, the Kabat collection, and the Protein Databank (Swindells M B et al., (2017) J Mol Biol. 2017 Feb. 3; 429(3):356-364).

The Kabat definition is based on sequence variability and is the commonly used (Kabat E A et al., supra).

In some aspects, the antibody or antigen-binding fragment thereof comprises the same complementarity determining regions (CDRs) as the CDRs found in SEQ ID NOs: 14 and 16.

In some aspects, the antibody or antigen-binding fragment thereof comprises the same complementarity determining regions (CDRs) as the CDRs found in any of the TCRs found herein.

In some aspects, the antibody or antigen-binding fragment thereof comprises the same variable regions as the variable regions found in any of the TCRs found herein.

In some aspects, the antibody or antigen-binding fragment thereof comprises a first variable region and a second variable region. In some aspects, the first variable region comprises an amino acid sequence corresponding to SEQ ID NO: 14. In some aspects, the first variable region comprises an amino acid sequence corresponding to SEQ ID NO: 16. In some aspects, the second variable region comprises an amino acid sequence corresponding to SEQ ID NO: 14. In some aspects, the second variable region comprises an amino acid sequence corresponding to SEQ ID NO: 16. In some aspects, the first variable region comprises an amino acid sequence corresponding to SEQ ID NO: 14 and the second variable region comprises an amino acid sequence corresponding to SEQ ID NO: 16. In some aspects, the first variable region comprises an amino acid sequence corresponding to SEQ ID NO: 16 and the second variable region comprises an amino acid sequence corresponding to SEQ ID NO: 14.

In some aspects, the antibody or antigen-binding fragment thereof comprises a variable region comprising an amino acid sequence having about 85%, about 90%, about 91% about 92%, about 93%, about 94%, about 95%, about 96%, about 97%, about 98%, about 99%, or about 100% sequence identity to SEQ ID NO: 14.

In some aspects, the antibody or antigen-binding fragment thereof comprises a variable region comprising an amino acid sequence having about 85%, about 90%, about 91% about 92%, about 93%, about 94%, about 95%, about 96%, about 97%, about 98%, about 99%, or about 100% sequence identity to SEQ ID NO: 16.

In some aspects, the antibody or antigen-binding fragment thereof comprises (i) a variable region (e.g., a first or second variable region) comprising an amino acid sequence having about 85%, about 90%, about 91% about 92%, about 93%, about 94%, about 95%, about 96%, about 97%, about 98%, about 99%, or about 100% sequence identity to SEQ ID NO: 14; and (ii) a variable region (e.g., a first or second variable region) comprising an amino acid sequence having about 85%, about 90%, about 91% about 92%, about 93%, about 94%, about 95%, about 96%, about 97%, about 98%, about 99%, or about 100% sequence identity to SEQ ID NO: 16.

In some aspects, the antibody or antigen-binding fragment thereof comprises (i) a variable region (e.g., a first or second variable region) comprising an amino acid sequence of SEQ ID NO: 14; and (ii) a variable region (e.g., a first or second variable region) comprising an amino acid sequence of SEQ ID NO: 16.

In some aspects, the antibody or antigen-binding fragment thereof is human, humanized, or chimeric.

In some aspects, the antibody or antigen-binding fragment thereof is an IgG antibody.

In some aspects, the IgG antibody is an IgG1 antibody or an IgG4 antibody.

In some aspects, the antibody is an antigen-binding fragment of an antibody.

In some aspects, said fragment is selected from the group consisting of Fab, F(ab′)2, Fv, scFv, scFv-Fc, dsFv and a single domain molecule.

In some aspects, said fragment is a scFv.

In some aspects, said fragment is a Fab.

In some aspects, said fragment is an intrabody.

In some aspects, the antigen-binding fragment is devoid of an Fc region.

In some aspects, an antibody or antigen-binding fragment thereof disclosed herein is an isolated antibody or antigen-binding fragment thereof. In some aspects, an antibody or antigen-binding fragment thereof disclosed herein is a monoclonal antibody or antigen-binding fragment thereof. In some aspects, an antibody or antigen-binding fragment thereof disclosed herein is not a polyclonal antibody or antigen-binding fragment thereof.

### VII. Methods of Treatment/Use

Certain aspects of the disclosure are directed to methods of killing a target cancer cell using the engineered TCRs, TCR T-cells, BiTE molecules, or antibodies or antigen binding fragment thereof disclosed herein. In particular, the disclosure provides TCR based therapies for patients harboring EGFR(T790M) mutation on HLA-A*02:01.

Cytotoxic T lymphocytes (CTLs) that recognize mutated peptides (neoantigens) presented at the tumor cell surface by HLA class I molecules represent powerful antitumor immune cells that are capable of causing regressions of large tumors in cancer patients. Engineered T-cell receptors (TCRs) derived from such tumor antigen-specific T cells as disclosed herein can be cloned and isolated to create TCR-based therapeutics with anti-cancer targeting ability, including engineered TCR-T cells or BiTE therapeutics.

As a natural consequence of high HLA diversity and the private nature of most tumor-associated mutations, very few neoantigen targets are shared between patients, and neoantigen-specific TCRs are very few in number and at very early stages of development in clinical trials. The therapies herein can be directed to engineered T-cell receptors that targets the shared EGFR mutation T790M mutation, which is expressed by >50% of patients progressing on first and second generation EGFR TKIs presented on the most prevalent HLA-A*02:01 allele in the United States (>40%). Given the high prevalence of EGFR mutations in lung cancer and the high prevalence of this HLA molecules in the human population, the engineered TCR based therapies disclosed herein can have therapeutic value for many lung cancer patients that have a T790M mutation on human leukocyte antigen-A (HLA-A)*02:01 (SEQ ID NO:1).

In some aspects, provided herein is a method of treating cancer in an individual, the method comprising administering to the individual a therapeutically effective amount of any of the epidermal growth factor receptor (EGFR) comprising a T790M mutation on human leukocyte antigen-A (HLA-A)*02:01 binding molecules (e.g., the engineered TCRs, TCR T-cells, BiTE molecules, or antibodies or antigen binding fragment thereof disclosed herein).

In some aspects, the cancer is a lung cancer.

In some aspects, wherein the lung cancer is non-small cell lung cancer (NSCLC).

In some aspects, the method comprises administering a therapeutically effective dose of the engineered TCRs, TCR T-cells, BiTE molecules, or antibodies or antigen binding fragment thereof disclosed herein to the subject parenthetically, intramuscularly, subcutaneously, ophthalmic, intravenously, intraperitoneally, intradermally, intraorbitally, intracerebrally, intracranially, intraspinally, intraventricular, intrathecally, intracistemally, intracapsularly, or intratumorally. In some aspects, the bispecific antibody is administered to the subject parenthetically, intramuscularly, subcutaneously, ophthalmic, intravenously, intraperitoneally, intradermally, intraorbitally, intracerebrally, intracranially, intraspinally, intraventricular, intrathecally, intracistemally, intracapsularly, or intratumorally. In certain aspects, the bispecific antibody is administered intratumorally.

The amount of an engineered TCRs or composition which will be effective in the treatment or prevention of a condition will depend on the nature of the disease. The precise dose to be employed in a composition will also depend on the route of administration, and the seriousness of the disease.

In some aspects, the subject has been or is currently being treated with an epidermal growth factor receptor (EGFR) tyrosine kinase inhibitor (EGFR-TKI). In some aspects, the cancer or tumor comprises an epidermal growth factor receptor (EGFR) T790M mutation on human leukocyte antigen-A (HLA-A)*02:01. In some aspects, the cancer or tumor is epidermal growth factor receptor (EGFR) tyrosine kinase inhibitor (EGFR-TKI) resistant.

### EXAMPLES

### Example 1. Analysis of Antigen-Specific T Cells from Healthy Donor Blood

Normal donor's peripheral blood mononuclear cells (PBMCs) were cultured with Dendritic Cell (DC) growth factor granulocyte-macrophage colony-stimulating factor (GM-CSF) and IL-4 for one week to get autologous dendritic cells.

After treatment with Tumor Necrosis Factor alpha (TNF-α), IL-6 and IL-1b for 24 hours, the matured DC were pulsed with T790M peptide. The peptide pulsed DC together with IL-21 were subsequently co-cultured with the PBMC from the same donor at (a ratio 1:35 of DC to PBMC) for one week to stimulate antigen specific T cell growth. The same procedure was then repeated twice.

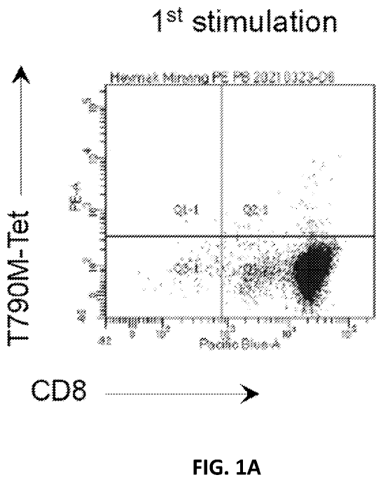

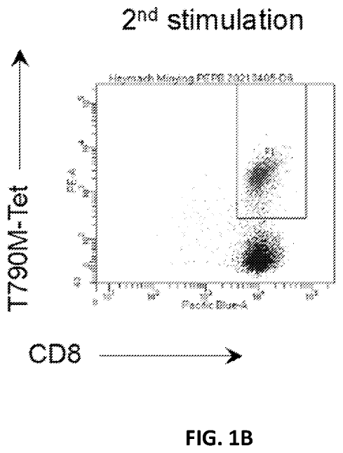

After 3 weeks of co-culture, T cells were then collected and stained with PE-labeled T790M tetramer and a CD8 antibody. The Tetramer/CD8 double positive T cells were then sorted by flow cytometry as shown in FIG. 1A.

Following DC stimulation and antigen specific T cell sorting, the number of T790M tetramer/CD8 double positive T cell consistently resulted in low volume.

To achieve a suitable level of T cells for a functional assay function and TCR identification, double positive T cells from the first stimulation and sorting were collected and utilized in rapid T cell amplification procedure by stimulation with anti-CD3 (OKT) antibody and IL-2 for two weeks.

The T cells were then stained and sorted again as shown in FIG. 1B. The tetramer/CD8 double positive T cells were used for both target cytotoxicity killing assay and TCR a/P chain determination.

### Example 2. Analysis of the Differences in Killing Ability of T Cells

To analyze T790M specific T cell activity, the lung cancer cell line H1975, which exhibits the endogenous T790M mutation, was stably transduced with HLAA0201 and used as a target for cytotoxicity killing assay assessment.

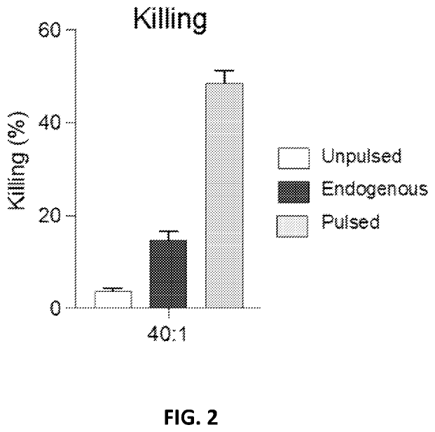

The H1975 parental cell, H1975A0201 cells were pulsed with the mutagenic peptide and a separate pulse of the H1975A0201 cell line occurred afterwards. Both cell lines were then appropriately labeled with chromium Cr51 for two hours, and co-cultured with T790M specific T cells for a total duration of 4 hours. The released Cr51 was then measured by a gamma counter and calculated for killing efficacy as shown in FIG. 2.

### Example 3. Isolated TCR Sequences

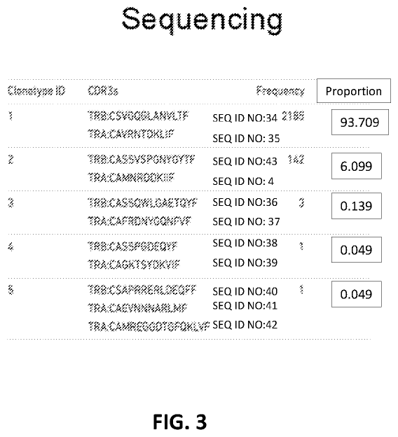

TCRs in T790M specific T cell population were determined by single T cell RNA sequencing using 10× Genomics Chromium Service at the MD Anderson DNA core facility. The TCR a/P pair sequence was analyzed using the Cell Ranger program and sequenced, as shown in FIG. 3.

### Example 4. TCR Transduction into Donor PBMCs

TCR a/P chains were then cloned into a retroviral vector pMSGV1 and stably transduced into normal donor's PBMC.

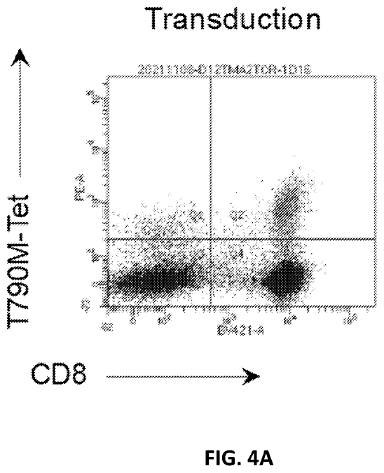

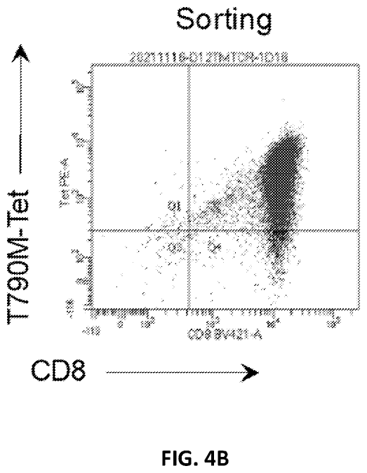

Following a 5 day transduction period, the T790M tetramer/CD8 double positive T cells were then sorted out for rapid T cell amplification (FIG. 4A). Sorting of the T790M TCR expression T cells after rapid T cell amplification then took place (FIG. 4B).

### Example 5. Transduced T Cells Targeting Tumor Cells Expressing the Antigen of Interest

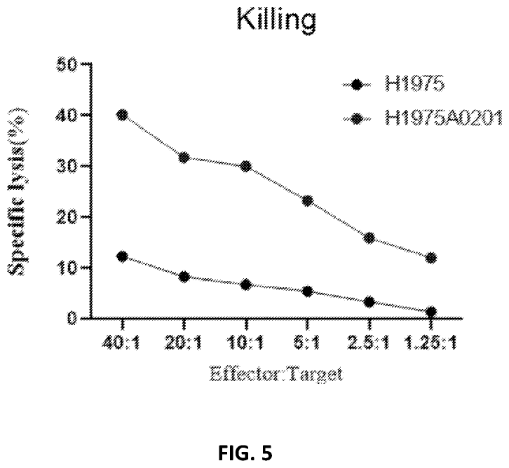

Cytotoxicity of T790M TCR expression T cells was then determined using methodology as described in Example 1. Chromium Cr51 labeled H1975 and H1975A0201 cells were then co-cultured with TCR-T cells at different effectors to target ratios, and killing activity was determined by measuring released Cr51 activity levels (FIG. 5).

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature.

All of the references cited above, as well as all references cited herein, are incorporated herein by reference in their entireties.

Any examples provided herein are offered by way of illustration and not by way of limitation.

What is claimed is:

1. An engineered T-cell Receptor (TCR) comprising:
(a) a TCR beta chain comprising (i) a variable TCR beta chain complementarity determining region 1 (CDR1β) comprising the amino acid sequence set forth in SEQ ID NO: 5, (ii) a CDR2β comprising the amino acid sequence set forth in SEQ ID NO: 6, and (iii) a CDR3β comprising the amino acid sequence set forth in SEQ ID NO: 7 or SEQ ID NO:43; and
(b) a TCR alpha chain comprising (i) a variable TCR alpha chain complementarity determining region 1 (CDR1α) comprising the amino acid sequence set forth in SEQ ID NO: 2, (ii) a CDR2α comprising the amino acid sequence set forth in SEQ ID NO: 3, and (iii) a CDR3α comprising the amino acid sequence set forth in SEQ ID NO: 4,
wherein the engineered TCR is capable of binding to an epitope of an epidermal growth factor receptor (EGFR) comprising a T790M mutation presented on a human leukocyte antigen-A (HLA-A)*02:01 antigen, wherein the epitope comprises the amino acid sequence of MQLMPFGCLL (SEQ ID NO: 1).

2. The engineered TCR of claim 1, wherein the TCR beta chain comprises any one of SEQ ID NO: 16, 32, or 46.

3. The engineered TCR of claim 1 or 2, wherein the TCR alpha chain comprises any one of SEQ ID NO: 14, 30, or 44.

4. The engineered TCR of any one of claims 1-3, wherein the TCR-β comprises an amino acid sequence having at least 85%, at least 90%, at least 91% at least 92%, at least 93%, at least 94%, at least 95%, at least 96%, at least 97%, at least 98%, at least 99%, or at least 100% sequence identity to SEQ ID NO: 16 and/or the TCR-α comprises an amino acid sequence having at least 85%, at least 90%, at least 91%, at least 92%, at least 93%, at least 94%, at least 95%, at least 96%, at least 97%, at least 98%, at least 99%, or at least 100% sequence identity to SEQ ID NO: 14.

5. The engineered TCR of any one of claims 1-3, wherein the TCR-β comprises an amino acid sequence having at least 85%, at least 90%, at least 91% at least 92%, at least 93%, at least 94%, at least 95%, at least 96%, at least 97%, at least 98%, at least 99%, or at least 100% sequence identity to SEQ ID NO: 46 and/or the TCR-α comprises an amino acid sequence having at least 85%, at least 90%, at least 91%, at least 92%, at least 93%, at least 94%, at least 95%, at least 96%, at least 97%, at least 98%, at least 99%, or at least 100% sequence identity to SEQ ID NO: 44.

6. The engineered TCR of any one of claims 1-3, wherein the TCR-β comprises an amino acid sequence having at least 85%, at least 90%, at least 91% at least 92%, at least 93%, at least 94%, at least 95%, at least 96%, at least 97%, at least 98%, at least 99%, or at least 100% sequence identity to SEQ ID NO: 32 and the TCR-α comprises an amino acid sequence having at least 85%, at least 90%, at least 91%, at least 92%, at least 93%, at least 94%, at least 95%, at least 96%, at least 97%, at least 98%, at least 99%, or at least 100% sequence identity to SEQ ID NO: 30.

7. The engineered TCR of any one of claims 1-3, wherein the TCR-β chain comprises an amino acid sequence having the amino acid sequence of SEQ ID NO: 32.

8. The engineered TCR of any one of claims 1-3, wherein the TCR-α chain comprises an amino acid sequence having the amino acid sequence of SEQ ID NO: 30.

9. The engineered TCR of any one of claims 1-8, wherein the engineered TCR is humanized or chimeric.

10. The engineered TCR of any one of claims 1-9, wherein the engineered TCR comprises an amino acid sequence having at least 85%, at least 90%, at least 91%, at least 92%, at least 93%, at least 94%, at least 95%, at least 96%, at least 97%, at least 98%, at least 99%, or at least 100% sequence identity to SEQ ID NO:28.

11. A polynucleotide comprising a nucleic acid encoding a TCR-β chain of any one of claims 1-10.

12. A polynucleotide comprising a nucleic acid encoding a TCR-β chain comprising a nucleic acid sequence having at least 85%, at least 90%, at least 91%, at least 92%, at least 93%, at least 94%, at least 95%, at least 96%, at least 97%, at least 98%, at least 99%, or at least 100% sequence identity to SEQ ID NO: 17, 47, or 33.

13. A polynucleotide comprising a nucleic acid encoding a TCR-α chain of any one of claims 1-10.

14. A polynucleotide comprising a nucleic acid encoding a TCR-α chain comprising a nucleic acid sequence having at least 85%, at least 90%, at least 91%, at least 92%, at least 93%, at least 94%, at least 95%, at least 96%, at least 97%, at least 98%, at least 99%, or at least 100% sequence identity to SEQ ID NO: 15, 45, or 31.

15. A polynucleotide comprising a nucleic acid encoding a TCR-β chain and a TCR-α chain of any one of claims 1-10.

16. A polynucleotide comprising a polynucleotide of claim 11 or 12 and polynucleotide of claim 13 or 14.

17. The polynucleotide of any one of claims 11-16, comprising a nucleic acid sequence having at least 85%, at least 90%, at least 91% at least 92%, at least 93%, at least 94%, at least 95%, at least 96%, at least 97%, at least 98%, at least 99%, or at least 100% sequence identity to SEQ ID NO: 29.

18. A vector comprising a polynucleotide of any one of claims 11-17.

19. A host cell comprising a polynucleotide of any one of claims 11-17 or a vector of claim 18.

20. A T-cell (TCR-T cell) comprising an engineered TCR of any one of claims 1-10.

21. The TCR-T cell of claim 20, wherein the T cell is a CD4+ T cell, a CD8+ T cell, or a CD4+/CD8+ T cell.

22. The TCR-T cell of claim 20 or 21, wherein the T cell is a natural killer (NK) cell.

23. The TCR-T cell of any one of claims 20-22, wherein T cells are isolated from peripheral blood mononuclear cells (PBMCs).

24. A bispecific T cell engager (BiTE) comprising an engineered TCR of any one of claims 1-10.

25. A method of treating cancer or a tumor in a subject, comprising administering to the subject a therapeutically effective amount of the engineered TCR, the TCR-T cell, or the BiTE of any one of the previous claims.

26. The method of claim 25, wherein the subject is a human.

27. The method of any one of claims 25-26, wherein the cancer is lung cancer.

28. The method of claim 27, wherein the lung cancer is non-small cell lung cancer (NSCLC).

29. The method of any one of claims 25-28, wherein the cancer or tumor comprises an epidermal growth factor receptor (EGFR) T790M mutation on human leukocyte antigen-A (HLA-A)*02:01.

30. The method of any one of claims 25-29, wherein the cancer or tumor is epidermal growth factor receptor (EGFR) tyrosine kinase inhibitor (EGFR-TKI) resistant.


SEQUENCE LISTING (47 sequences)

SEQ ID NO: 1 - protein, 10 aa, synthetic construct
  REGION 1..10: note = epidermal growth factor receptor (EGFR) comprising a T790Mmutation on human leukocyte antigen-A (HLA-A)*02:01 antigen
MQLMPFGCLL

SEQ ID NO: 2 - protein, 6 aa, synthetic construct
  REGION 1..6: note = TCR A CDR 1
NSAFQY

SEQ ID NO: 3 - protein, 6 aa, synthetic construct
  REGION 1..6: note = TCR A CDR 2
TYSSGN

SEQ ID NO: 4 - protein, 11 aa, synthetic construct
  REGION 1..11: note = TCR A CDR 3
CAMNRDDKIIF

SEQ ID NO: 5 - protein, 5 aa, synthetic construct
  REGION 1..5: note = TCR B CDR 1
MNHNS

SEQ ID NO: 6 - protein, 6 aa, synthetic construct
  REGION 1..6: note = TCR B CDR 2
SASEGT

SEQ ID NO: 7 - protein, 12 aa, synthetic construct
  REGION 1..12: note = TCR B CDR 3
ASSVSPGNYGYT

SEQ ID NO: 8 - DNA, 18 bp, synthetic construct
  source 1..18: mol_type = other DNA
  misc_feature 1..18: note = TCR A CDR 1
aacagtgcttttcaatac

SEQ ID NO: 9 - DNA, 18 bp, synthetic construct
  source 1..18: mol_type = other DNA
  misc_feature 1..18: note = TCR A CDR 2
acatactccagtggtaac

SEQ ID NO: 10 - DNA, 27 bp, synthetic construct
  source 1..27: mol_type = other DNA
  misc_feature 1..27: note = TCR A CDR 3
gcaatgaatcgggatgacaagatcatc

SEQ ID NO: 11 - DNA, 15 bp, synthetic construct
  source 1..15: mol_type = other DNA
  misc_feature 1..15: note = TCR B CDR 1
atgaaccataactcc

SEQ ID NO: 12 - DNA, 18 bp, synthetic construct
  source 1..18: mol_type = other DNA
  misc_feature 1..18: note = TCR B CDR 2
tcagcttctgagggtacc

SEQ ID NO: 13 - DNA, 36 bp, synthetic construct
  source 1..36: mol_type = other DNA
  misc_feature 1..36: note = TCR B CDR 3
gccagcagtgtttcccctggaaactatggctacacc

SEQ ID NO: 14 - protein, 110 aa, synthetic construct
  REGION 1..110: note = TCR A Variable Chain
QQKEVEQDPGPLSVPEGAIVSLNCTYSNSAFQYFMWYRQYSRKGPELLMYTYSSGNKEDGRFTAQVDKSSKYISLFIRDSQPSDSATYLCAMNRDDKIIFGKGTRLHILP

SEQ ID NO: 15 - DNA, 328 bp, synthetic construct
  source 1..328: mol_type = other DNA
  misc_feature 1..328: note = TCR A Variable Chain
cagaaggaggtggagcaggatcctggaccactcagtgttccagagggagccattgtttctctcaactgcacttacagcaacagtgcttttcaatacttcatgtggtacagacagtattccagaaaaggccctgagttgctgatgtacacatactccagtggtaacaaagaagatggaaggtttacagcacaggtcgataaatccagcaagtatatctccttgttcatcagagactcacagcccagtgattcagccacctacctctgtgcaatgaatcgggatgacaagatcatctttggaaaagggacacgacttcatattctcccca

SEQ ID NO: 16 - protein, 111 aa, synthetic construct
  REGION 1..111: note = TCR B Variable Chain
GVTQTPKFQVLKTGQSMTLQCAQDMNHNSMYWYRQDPGMGLRLIYYSASEGTTDKGEVPNGYNVSRLNKREFSLRLESAAPSQTSVYFCASSVSPGNYGYTFGSGTRLTVV

SEQ ID NO: 17 - DNA, 340 bp, synthetic construct
  source 1..340: mol_type = other DNA
  misc_feature 1..340: note = TCR B Variable Chain
aatgctggtgtcactcagaccccaaaattccaggtcctgaagacaggacagagcatgacactgcagtgtgcccaggatatgaaccataactccatgtactggtatcgacaagacccaggcatgggactgaggctgatttattactcagcttctgagggtaccactgacaaaggagaagtccccaatggctacaatgtctccagattaaacaaacgggagttctcgctcaggctggagtcggctgctccctcccagacatctgtgtacttctgtgccagcagtgtttcccctggaaactatggctacaccttcggttcggggaccaggttaaccgttgtag

SEQ ID NO: 18 - protein, 141 aa, synthetic construct
  REGION 1..141: note = Alpha chain constant region
NIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 19 - DNA, 425 bp, synthetic construct
  source 1..425: mol_type = other DNA
  misc_feature 1..425: note = Alpha chain constant region
atatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgcgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 20 - protein, 177 aa, synthetic construct
  REGION 1..177: note = Beta Chain constant region
EDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 21 - DNA, 530 bp, synthetic construct
  source 1..530: mol_type = other DNA
  misc_feature 1..530: note = Beta Chain constant region
aggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtctgcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 22 - protein, 27 aa, synthetic construct
  REGION 1..27: note = Linker
RAKRSGSGATNFSLLKQAGDVEENPGP

SEQ ID NO: 23 - DNA, 81 bp, synthetic construct
  source 1..81: mol_type = other DNA
  misc_feature 1..81: note = Linker
cgtgccaagcgaagcggatctggcgccacgaacttctctctgttaaagcaagcaggagatgttgaagaaaaccccgggcct

SEQ ID NO: 24 - protein, 21 aa, synthetic construct
  REGION 1..21: note = Signal peptide
MSIGLLCCVAFSLLWASPVNA

SEQ ID NO: 25 - protein, 20 aa, synthetic construct
  REGION 1..20: note = Signal peptide
MKSLRVLLVILWLQLSWVWS

SEQ ID NO: 26 - DNA, 57 bp, synthetic construct
  source 1..57: mol_type = other DNA
  misc_feature 1..57: note = Signal peptide
atgagcatcgggctcctgtgctgtgtggccttttctctcctgtgggcaagtccagtg

SEQ ID NO: 27 - DNA, 63 bp, synthetic construct
  source 1..63: mol_type = other DNA
  misc_feature 1..63: note = Signal peptide
atgaaatccttgagagttttactggtgatcctgtggcttcagttaagctgggtttggagccaa

SEQ ID NO: 28 - protein, 607 aa, synthetic construct
  REGION 1..607: note = full TCR sequence
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASSVSPGNYGYTFGSGTRLTVVEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFRAKRSGSGATNFSLLKQAGDVEENPGPMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMNRDDKIIFGKGTRLHILPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 29 - DNA, 1824 bp, synthetic construct
  source 1..1824: mol_type = other DNA
  misc_feature 1..1824: note = full TCR sequence
atgagcatcgggctcctgtgctgtgtggccttttctctcctgtgggcaagtccagtgaatgctggtgtcactcagaccccaaaattccaggtcctgaagacaggacagagcatgacactgcagtgtgcccaggatatgaaccataactccatgtactggtatcgacaagacccaggcatgggactgaggctgatttattactcagcttctgagggtaccactgacaaaggagaagtccccaatggctacaatgtctccagattaaacaaacgggagttctcgctcaggctggagtcggctgctccctcccagacatctgtgtacttctgtgccagcagtgtttcccctggaaactatggctacaccttcggttcggggaccaggttaaccgttgtagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtctgcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttccgtgccaagcgaagcggatctggcgccacgaacttctctctgttaaagcaagcaggagatgttgaagaaaaccccgggcctatgaaatccttgagagttttactggtgatcctgtggcttcagttaagctgggtttggagccaacagaaggaggtggagcaggatcctggaccactcagtgttccagagggagccattgtttctctcaactgcacttacagcaacagtgcttttcaatacttcatgtggtacagacagtattccagaaaaggccctgagttgctgatgtacacatactccagtggtaacaaagaagatggaaggtttacagcacaggtcgataaatccagcaagtatatctccttgttcatcagagactcacagcccagtgattcagccacctacctctgtgcaatgaatcgggatgacaagatcatctttggaaaagggacacgacttcatattctccccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgcgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 30 - protein, 271 aa, synthetic construct
  REGION 1..271: note = Full Alpha Chain
MKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMNRDDKIIFGKGTRLHILPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 31 - DNA, 816 bp, synthetic construct
  source 1..816: mol_type = other DNA
  misc_feature 1..816: note = Full Alpha Chain Nucleotide
atgaaatccttgagagttttactggtgatcctgtggcttcagttaagctgggtttggagccaacagaaggaggtggagcaggatcctggaccactcagtgttccagagggagccattgtttctctcaactgcacttacagcaacagtgcttttcaatacttcatgtggtacagacagtattccagaaaaggccctgagttgctgatgtacacatactccagtggtaacaaagaagatggaaggtttacagcacaggtcgataaatccagcaagtatatctccttgttcatcagagactcacagcccagtgattcagccacctacctctgtgcaatgaatcgggatgacaagatcatctttggaaaagggacacgacttcatattctccccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgcgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 32 - protein, 309 aa, synthetic construct
  REGION 1..309: note = Full Beta Chain Amino Acid
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASSVSPGNYGYTFGSGTRLTVVEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 33 - DNA, 927 bp, synthetic construct
  source 1..927: mol_type = other DNA
  misc_feature 1..927: note = Full Beta Chain Nucleotide
atgagcatcgggctcctgtgctgtgtggccttttctctcctgtgggcaagtccagtgaatgctggtgtcactcagaccccaaaattccaggtcctgaagacaggacagagcatgacactgcagtgtgcccaggatatgaaccataactccatgtactggtatcgacaagacccaggcatgggactgaggctgatttattactcagcttctgagggtaccactgacaaaggagaagtccccaatggctacaatgtctccagattaaacaaacgggagttctcgctcaggctggagtcggctgctccctcccagacatctgtgtacttctgtgccagcagtgtttcccctggaaactatggctacaccttcggttcggggaccaggttaaccgttgtagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtctgcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 34 - protein, 13 aa, synthetic construct
  REGION 1..13: note = Clonotype ID 1 TCR B CDR3
CSVGQGLANVLTF

SEQ ID NO: 35 - protein, 11 aa, synthetic construct
  REGION 1..11: note = Clonotype ID 1 TCR A CDR3
CAVRNTDKLIF

SEQ ID NO: 36 - protein, 14 aa, synthetic construct
  REGION 1..14: note = Clonotype ID 3 TCR B CDR3
CASSQWLGAETQYF

SEQ ID NO: 37 - protein, 13 aa, synthetic construct
  REGION 1..13: note = Clonotype ID 3 TCR A CDR3
CAFRDNYGQNFVF

SEQ ID NO: 38 - protein, 11 aa, synthetic construct
  REGION 1..11: note = Clonotype ID 4 TCR B CDR3
CASSPGDEQYF

SEQ ID NO: 39 - protein, 12 aa, synthetic construct
  REGION 1..12: note = Clonotype ID 4 TCR A CDR3
CAGKTSYDKVIF

SEQ ID NO: 40 - protein, 14 aa, synthetic construct
  REGION 1..14: note = Clonotype ID 5 TCR B CDR3
CSAPRRERLDEQFF

SEQ ID NO: 41 - protein, 12 aa, synthetic construct
  REGION 1..12: note = Clonotype ID 5 TCR A CDR3
CAEVNNNARLMF

SEQ ID NO: 42 - protein, 16 aa, synthetic construct
  REGION 1..16: note = Clonotype ID 5 TCR A CDR3
CAMREGGDTGFQKLVF

SEQ ID NO: 43 - protein, 14 aa, synthetic construct
  REGION 1..14: note = Clonotype ID 2 TCR B CDR3
CASSVSPGNYGYTF

SEQ ID NO: 44 - protein, 251 aa, synthetic construct
  REGION 1..251: note = Alpha Chain Constant and Variable Region
QQKEVEQDPGPLSVPEGAIVSLNCTYSNSAFQYFMWYRQYSRKGPELLMYTYSSGNKEDGRFTAQVDKSSKYISLFIRDSQPSDSATYLCAMNRDDKIIFGKGTRLHILPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 45 - protein, 753 aa, synthetic construct
  REGION 1..753: note = Alpha Chain Constant and Variable Region
CAGAAGGAGGTGGAGCAGGATCCTGGACCACTCAGTGTTCCAGAGGGAGCCATTGTTTCTCTCAACTGCACTTACAGCAACAGTGCTTTTCAATACTTCATGTGGTACAGACAGTATTCCAGAAAAGGCCCTGAGTTGCTGATGTACACATACTCCAGTGGTAACAAAGAAGATGGAAGGTTTACAGCACAGGTCGATAAATCCAGCAAGTATATCTCCTTGTTCATCAGAGACTCACAGCCCAGTGATTCAGCCACCTACCTCTGTGCAATGAATCGGGATGACAAGATCATCTTTGGAAAAGGGACACGACTTCATATTCTCCCCAATATCCAGAACCCTGACCCTGCCGTGTACCAGCTGAGAGACTCTAAATCCAGTGACAAGTCTGTCTGCCTATTCACCGATTTTGATTCTCAAACAAATGTGTCACAAAGTAAGGATTCTGATGTGTATATCACAGACAAATGCGTGCTAGACATGAGGTCTATGGACTTCAAGAGCAACAGTGCTGTGGCCTGGAGCAACAAATCTGACTTTGCATGTGCAAACGCCTTCAACAACAGCATTATTCCAGAAGACACCTTCTTCCCCAGCCCAGAAAGTTCCTGTGATGTCAAGCTGGTCGAGAAAAGCTTTGAAACAGATACGAACCTAAACTTTCAAAACCTGTCAGTGATTGGGTTCCGAATCCTCCTCCTGAAAGTGGCCGGGTTTAATCTGCTCATGACGCTGCGGCTGTGGTCCAGCTGA

SEQ ID NO: 46 - protein, 288 aa, synthetic construct
  REGION 1..288: note = Beta Chain Constant and Variable Region
GVTQTPKFQVLKTGQSMTLQCAQDMNHNSMYWYRQDPGMGLRLIYYSASEGTTDKGEVPNGYNVSRLNKREFSLRLESAAPSQTSVYFCASSVSPGNYGYTFGSGTRLTVVEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 47 - protein, 870 aa, synthetic construct
  REGION 1..870: note = Beta Chain Constant and Variable Region
AATGCTGGTGTCACTCAGACCCCAAAATTCCAGGTCCTGAAGACAGGACAGAGCATGACACTGCAGTGTGCCCAGGATATGAACCATAACTCCATGTACTGGTATCGACAAGACCCAGGCATGGGACTGAGGCTGATTTATTACTCAGCTTCTGAGGGTACCACTGACAAAGGAGAAGTCCCCAATGGCTACAATGTCTCCAGATTAAACAAACGGGAGTTCTCGCTCAGGCTGGAGTCGGCTGCTCCCTCCCAGACATCTGTGTACTTCTGTGCCAGCAGTGTTTCCCCTGGAAACTATGGCTACACCTTCGGTTCGGGGACCAGGTTAACCGTTGTAGAGGACCTGAACAAGGTGTTCCCACCCGAGGTCGCTGTGTTTGAGCCATCAGAAGCAGAGATCTCCCACACCCAAAAGGCCACACTGGTGTGCCTGGCCACAGGCTTCTTCCCTGACCACGTGGAGCTGAGCTGGTGGGTGAATGGGAAGGAGGTGCACAGTGGGGTCTGCACGGACCCGCAGCCCCTCAAGGAGCAGCCCGCCCTCAATGACTCCAGATACTGCCTGAGCAGCCGCCTGAGGGTCTCGGCCACCTTCTGGCAGAACCCCCGCAACCACTTCCGCTGTCAAGTCCAGTTCTACGGGCTCTCGGAGAATGACGAGTGGACCCAGGATAGGGCCAAACCCGTCACCCAGATCGTCAGCGCCGAGGCCTGGGGTAGAGCAGACTGTGGCTTTACCTCGGTGTCCTACCAGCAAGGGGTCCTGTCTGCCACCATCCTCTATGAGATCCTGCTAGGGAAGGCCACCCTGTATGCTGTGCTGGTCAGCGCCCTTGTGTTGATGGCCATGGTCAAGAGAAAGGATTTC